(19) United States
(12) Patent Application Publication
(10) Pub. No.: US 2022/0163532 A1
(43) Pub. Date: May 26, 2022

(54) NUCLEAR-DERIVED EXOSOMES AND METHODS OF USE THEREOF

(71) Applicant: Board of Regents, The University of Texas System, Austin, TX (US)

(72) Inventors: Anil K. SOOD, Houston, TX (US); Akira YOKOI, Houston, TX (US)

(73) Assignee: Board of Regents, The University of Texas System, Austin, TX (US)

(21) Appl. No.: 17/598,780

(22) PCT Filed: Mar. 27, 2020
(86) PCT No.: PCT/US2020/025353

§ 371 Date: Sep. 27, 2021

Related U.S. Application Data
(60) Provisional application No. 62/824,581, filed on Mar. 27, 2019.

(51) Int. Cl. G01N 33/574

(52) U.S. Cl. CPC G01N 33/57449

(57) ABSTRACT

The present invention provides that exosomes from ovarian cancer patients contain nuclear proteins and genomic DNA at an increased proportion. As such, detecting nuclear-derived exosomes provides a method of early detection of ovarian cancer. Furthermore, the level of nuclear-derived exosomes can be monitored over time to assess responsiveness to genotoxic therapy.

### REFERENCE TO RELATED APPLICATIONS

The present application claims the priority benefit of U.S. provisional application No. 62/824,581, filed Mar. 27, 2019, the entire contents of which is incorporated herein by reference.

### STATEMENT REGARDING FEDERALLY SPONSORED RESEARCH

This invention was made with government support under Grant No. CA209904 awarded by the National Institutes of Health. The government has certain rights in the invention.

### REFERENCE TO A SEQUENCE LISTING

The instant application contains a Sequence Listing, which has been submitted in ASCII format via EFS-Web and is hereby incorporated by reference in its entirety. Said ASCII copy, created on Mar. 27, 2020, is named UTFCP1453WP_ST25.txt and is 0.9 kilobytes in size.

### BACKGROUND

### 1. Field

The present invention relates generally to the fields of medicine and oncology. More particularly, it concerns methods of assays the nuclear content of exosomes in order to detect ovarian cancer and assess responsiveness to genotoxic therapy.

### 2. Description of Related Art

Exosomes are small extracellular vesicles secreted from all living cells that mediate cell to cell communication as well as a plethora of biological and cellular functions (Thery et al., 2002). The contents of exosomes are highly varied, including proteins, small RNAs and recently discovered, genomic DNA (gDNA) (Thakur et al., 2014). The presence of nuclear content in exosomes suggests that different cell compartments can contribute to exosome cargo; however, little is known about this subpopulation of nuclear derived exosomes (nExo). The clinical relevance, such as cancer detection and therapeutic response monitoring, of nExo remains to be discovered.

### SUMMARY

As such, in one embodiment, provided herein are methods of diagnosing cancer in a patient, the method comprising: (a) obtaining a body fluid sample from a patient;

(b) isolating an exosomes fraction of the body fluid sample; and (c) assaying for the presence of nuclear proteins and/or genomic DNA in the exosomes fraction, wherein if nuclear proteins and/or genomic DNA is present, then the patient is diagnosed as having cancer.

In some aspects, the methods further comprise quantifying the number of nuclear proteins and/or genomic DNA-containing exosomes in the patient. In some aspects, the number of nuclear proteins and/or genomic DNA-containing exosomes in the patient is higher than a reference number. In some aspects, the reference number is the number of nuclear proteins and/or genomic DNA-containing exosomes in a sample obtained from a healthy subject.

In some aspects, the methods are further defined as methods of monitoring response to a therapy in a cancer patient, wherein if the number of nuclear proteins and/or genomic DNA-containing exosomes increases over time, then the patient is said to have had a positive response to the therapy. In some aspects, the therapy is a genotoxic therapy. In some aspects, the genotoxic therapy is a PARP inhibitor (e.g., olaparib, rucaparib, niraparib, talazoparib, veliparib, pamiparib, CEP 9722, E7016, iniparib) or a topoisomerase inhibitor (e.g., topotecan, irinotecan, camptothecin, lamellarin D, etoposide, teniposide, doxorubicin, daunorubicin, mitoxantrone, amsacrine, ellipticines, aurintricarboxylic acid, HU-311, EGCG, genistein, quercetin, resveratrol).

In some aspects, the patient has not been previously diagnosed with cancer and the method is a method of early cancer detection. In some aspects, the patient is in remission and the method is a method of detecting relapse. In some aspects, the methods further comprise administering an anti-cancer therapy to the patient if the patient is diagnosed with cancer. In some aspects, the body fluid sample is ascites, serum, lymph, saliva, urine, cerebrospinal fluid, bone marrow aspirates, eye exudate/tears, or serum. In some aspects, the cancer is a ovarian cancer, breast cancer, lung cancer, head & neck cancer, prostate cancer, esophageal cancer, tracheal cancer, brain cancer, liver cancer, bladder cancer, stomach cancer, pancreatic cancer, uterine cancer, cervical cancer, testicular cancer, colon cancer, rectal cancer or skin cancer.

In some aspects, the methods further comprise reporting the diagnosis of the patient. In some aspects, reporting comprises preparing a written or electronic report. In some aspects, the methods further comprise providing the report to the patient, a doctor, a hospital or an insurance company.

In one embodiment, provided herein are methods of identifying genetic alterations in a tumor, the methods comprising: (a) obtaining an ascites fluid sample from a patient having a tumor; (b) isolating an exosomes fraction of the ascites fluid sample; and (c) assaying for the presence of genetic alterations in a genomic DNA present in the exosomes fraction, wherein any genetic alternations present in the genomic DNA present in the exosomes fraction are identifies as genetic alternation in the tumor. In some aspects, the methods further comprise administering an anti-cancer therapy to the patient if the patient. In some aspects, the cancer is a ovarian cancer, breast cancer, lung cancer, head & neck cancer, prostate cancer, esophageal cancer, tracheal cancer, brain cancer, liver cancer, bladder cancer, stomach cancer, pancreatic cancer, uterine cancer, cervical cancer, testicular cancer, colon cancer, rectal cancer or skin cancer.

In one embodiment, provided herein are methods of treating a cancer in a subject comprising, identifying a subject as having a cancer in accordance with any one of the present embodiments and administering an anti-cancer therapy to the subject. In some aspects, the anti-cancer therapy is a chemotherapy, a radiation therapy, a hormonal therapy, a targeted therapy, an immunotherapy or a surgical therapy. In some aspects, the subject is a human.

In one embodiment, provided herein are methods of selecting a cancer patient for treatment with a PARP inhibitor, the method comprising: (a) obtaining a body fluid sample from a patient; (b) isolating an exosomes fraction of the body fluid sample; and (c) assaying for the presence of PARP protein in the exosomes fraction, wherein if PARP protein is present, then the patient is selected for treatment with the PARP inhibitor. In some aspects, the patient has ovarian cancer.

In one embodiment, provided herein are methods of monitoring response to a therapy in a cancer patient, the method comprising: (a) obtaining a body fluid sample from a patient at two or more time points during the course of therapy; (b) isolating an exosomes fraction of the body fluid samples; and (c) assaying for the presence of nuclear proteins and/or genomic DNA in the exosomes fractions, wherein if the number of nuclear proteins and/or genomic DNA-containing exosomes increases over time, then the patient is said to have had a positive response to the therapy.

In some aspects, the therapy is a genotoxic therapy. In some aspects, the genotoxic therapy is a PARP inhibitor (e.g., olaparib, rucaparib, niraparib, talazoparib, veliparib, pamiparib, CEP 9722, E7016, iniparib) or a topoisomerase inhibitor (e.g., topotecan, irinotecan, camptothecin, lamellarin D, etoposide, teniposide, doxorubicin, daunorubicin, mitoxantrone, amsacrine, ellipticines, aurintricarboxylic acid, HU-311, EGCG, genistein, quercetin, resveratrol). In some aspects, the methods further comprise administering a different anti-cancer therapy to the patient if the patient is not found to have had a positive response to the therapy. In some aspects, the body fluid sample is ascites, serum, lymph, saliva, urine, cerebrospinal fluid, bone marrow aspirates, eye exudate/tears, or serum. In some aspects, the cancer is a ovarian cancer, breast cancer, lung cancer, head & neck cancer, prostate cancer, esophageal cancer, tracheal cancer, brain cancer, liver cancer, bladder cancer, stomach cancer, pancreatic cancer, uterine cancer, cervical cancer, testicular cancer, colon cancer, rectal cancer or skin cancer.

In one embodiment, provided herein are methods of analyzing exosomes, the methods comprising (a) obtaining a fluid sample comprising exosomes; (b) staining the exosomes with a plasma membrane stain; and (c) analyzing the exosomes using imaging flow cytometry by selecting particles with a side-scatter aspect ratio of about one. In some aspects, the exosomes are isolated between steps (a) and (b). In some aspects, the exosomes are not isolated between steps (a) and (b). In some aspects, the method is a method of isolating exosomes. In some aspects, the plasma membrane stain is a CellMask plasma membrane stain. In some aspects, the exosomes are also stained for the presence of genomic DNA and/or a nuclear protein.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, the variation that exists among the study subjects, or a value that is within 10% of a stated value.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1J. Characterization of nuclear-derived content from ovarian cancer exosomes. (FIG. 1A) TCGA pan-cancer ploidy analysis of 20 cancer types. n=62 [kidney chromophobe (KICH)], n=418 [brain low-grade glioma (LGG)], n=7 [pancreatic cancer (PAAD)], n=138 [pheochromocytoma (PCPG)], n=353 [prostate adenocarcinoma (PRAD)], n=184 [thyroid carcinoma (THCA)], n=543 [glioblastoma (GBM)], n=415 [kidney clear cell carcinoma (KIRC)], n=61 [uveal melanoma (UVM)], n=415 [uterine endometrial carcinoma (UCEC)], n=257 [skin cutaneous melanoma (SKCM)], n=501 [head and neck squamous carcinoma (HNSC)], n=155 [kidney papillary carcinoma (KIRP)], n=330 [stomach adenocarcinoma (STAD)], n=940 [breast cancer (BRCA)], n=187 [liver hepatocellular carcinoma (LIHC)], n=396 [colon adenocarcinoma (COAD)], n=34 [cervical cancer (CESC)], n=85 [adrenocortical carcinoma (ACC)], n=158 [renal adenocarcinoma (READ)], n=435 [lung squamous carcinoma (LU.S. C)], n=544 [ovarian cancer (OV)], n=429 [lung adenocarcinoma (LUAD)], n=144 [bladder cancer (BLCA)], and n=55 [uterine carcinosarcoma (UCS)]. (FIG. 1B) Cryo-EM image of the exosomes isolated from OVCAR-5 cells. Scale bars, 100 nm. (FIG. 1C) NTA for the exosomes isolated from OVCAR-5 cells. (FIG. 1D) Western blot analysis of exosome markers in OVCAR-5. TSG101, Alix, and CD63 are used as exosome markers, and GRP94 is used as a marker of cellular contamination. TCL, total cell lysate. (FIG. 1E) Pie chart of cellular compartment proteins resulting from MS analysis in OVCAR-5 cell—derived exosomes. Nuclear components are highlighted: 1, endoplasmic reticulum; 2, endosome; 3, Golgi; 4, cell surface;

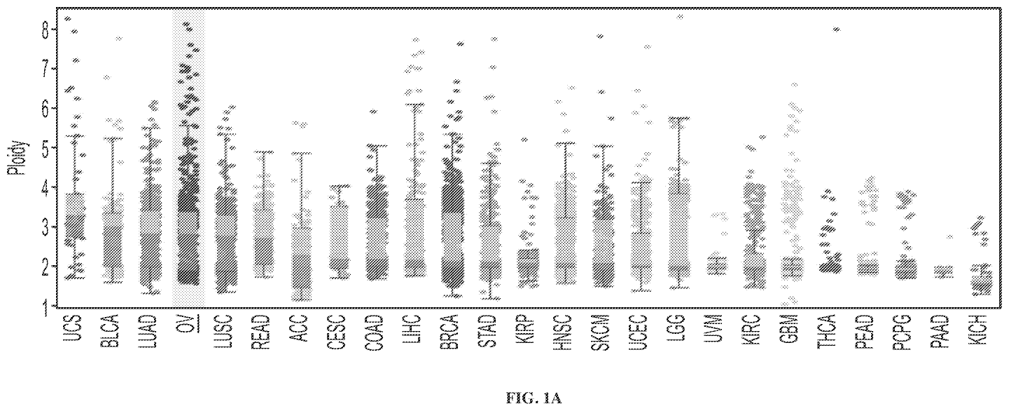

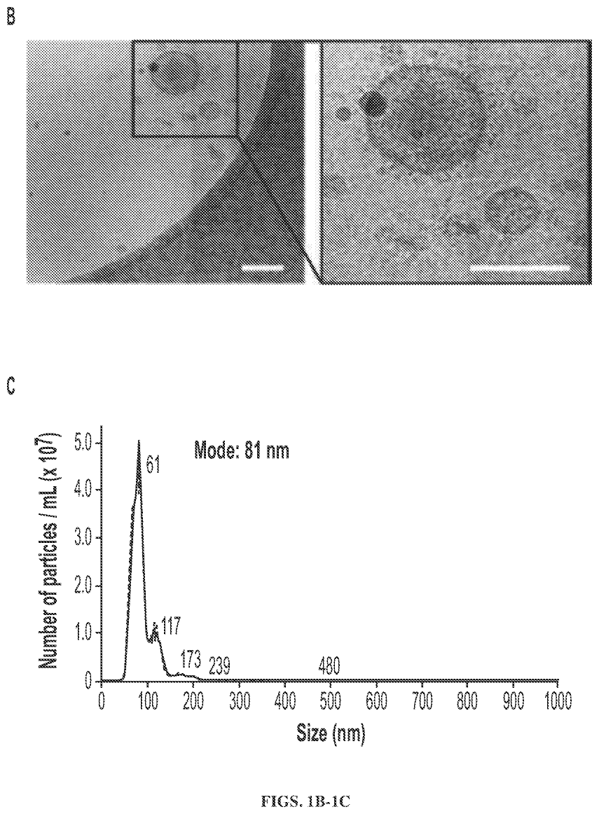

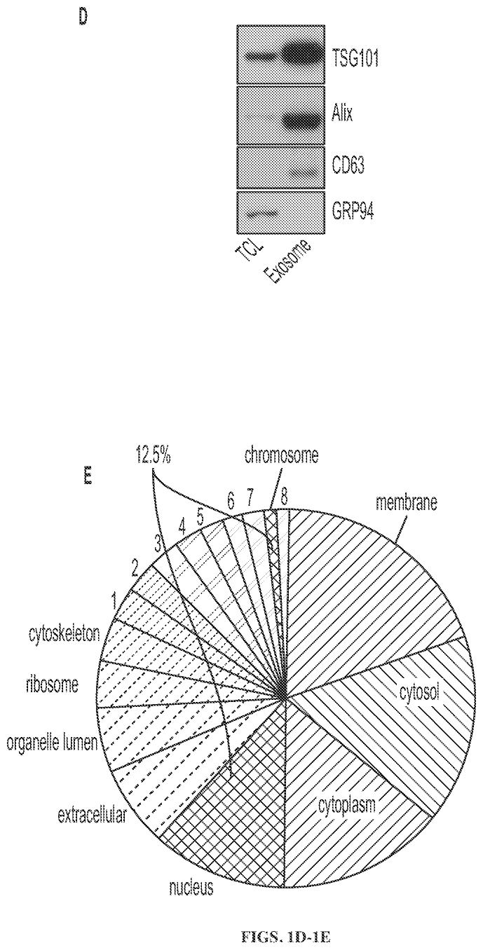

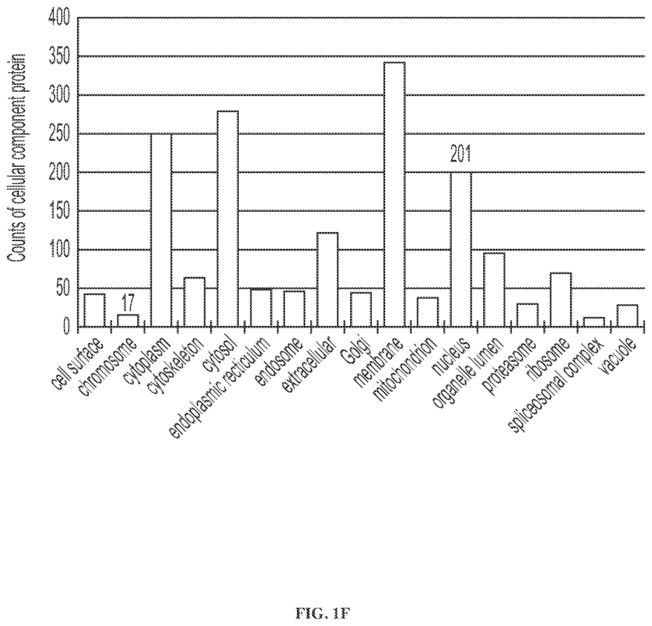

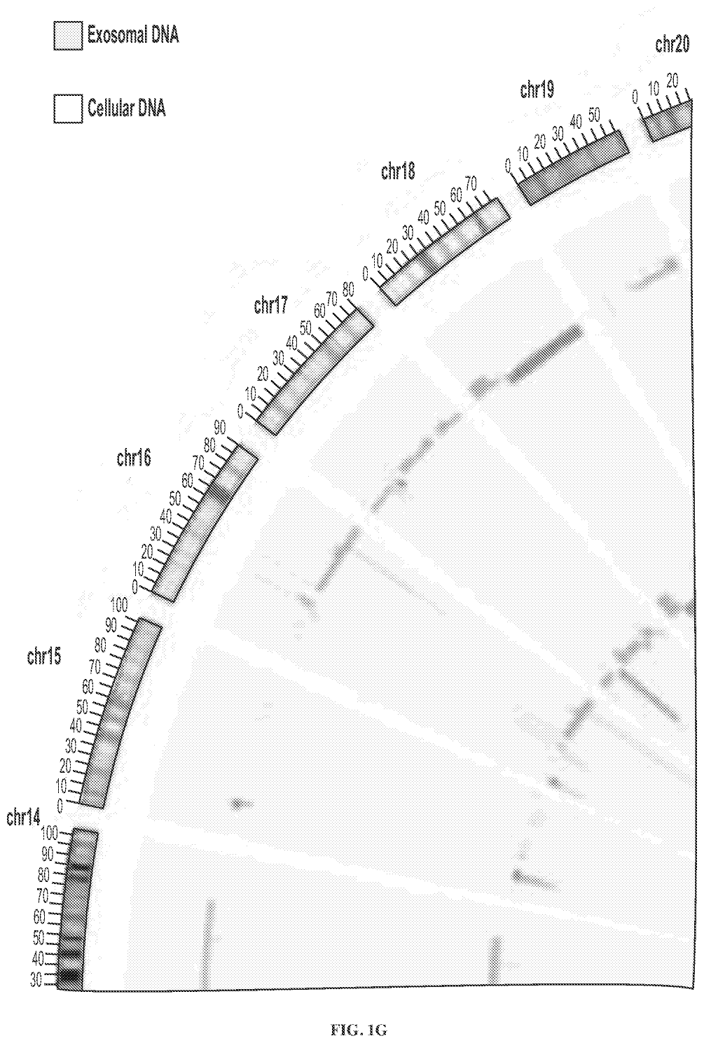

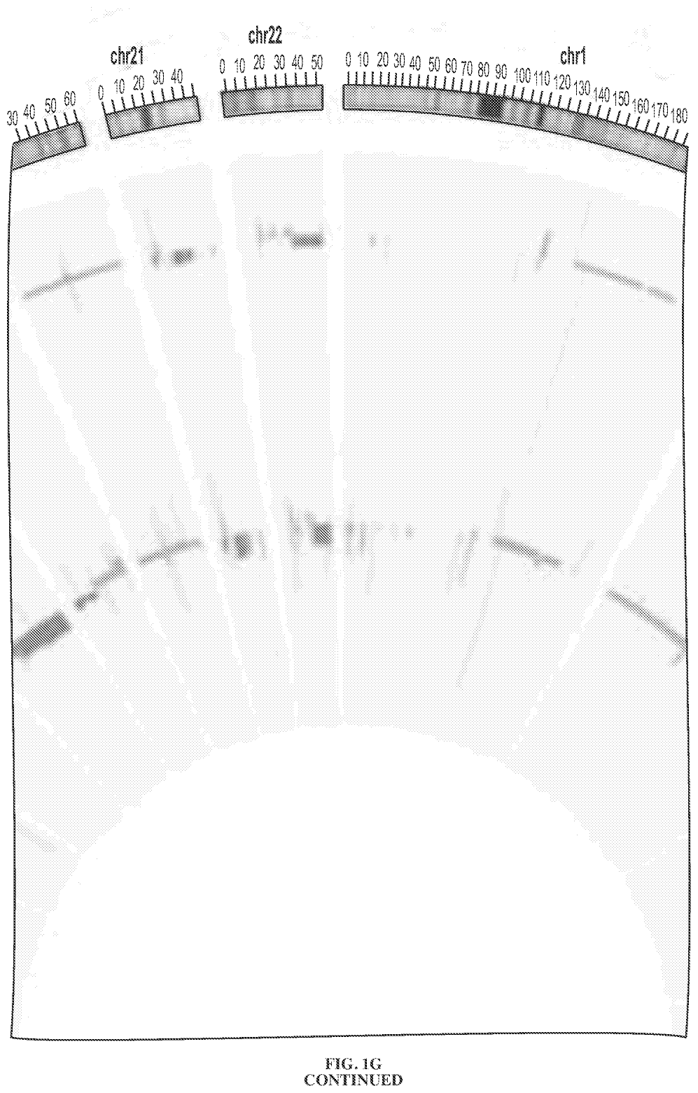

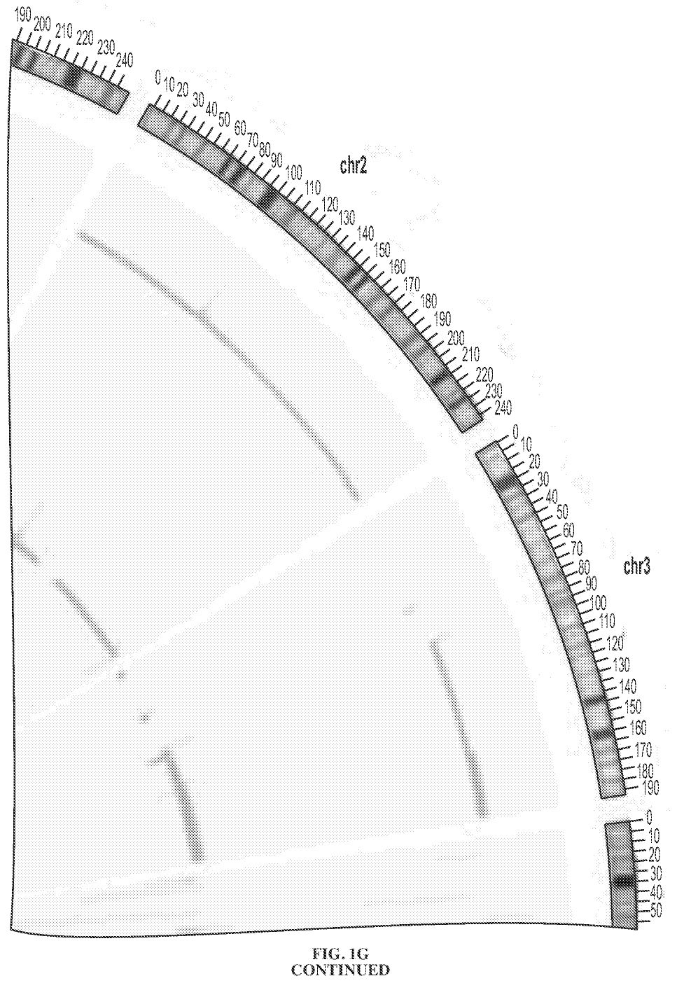

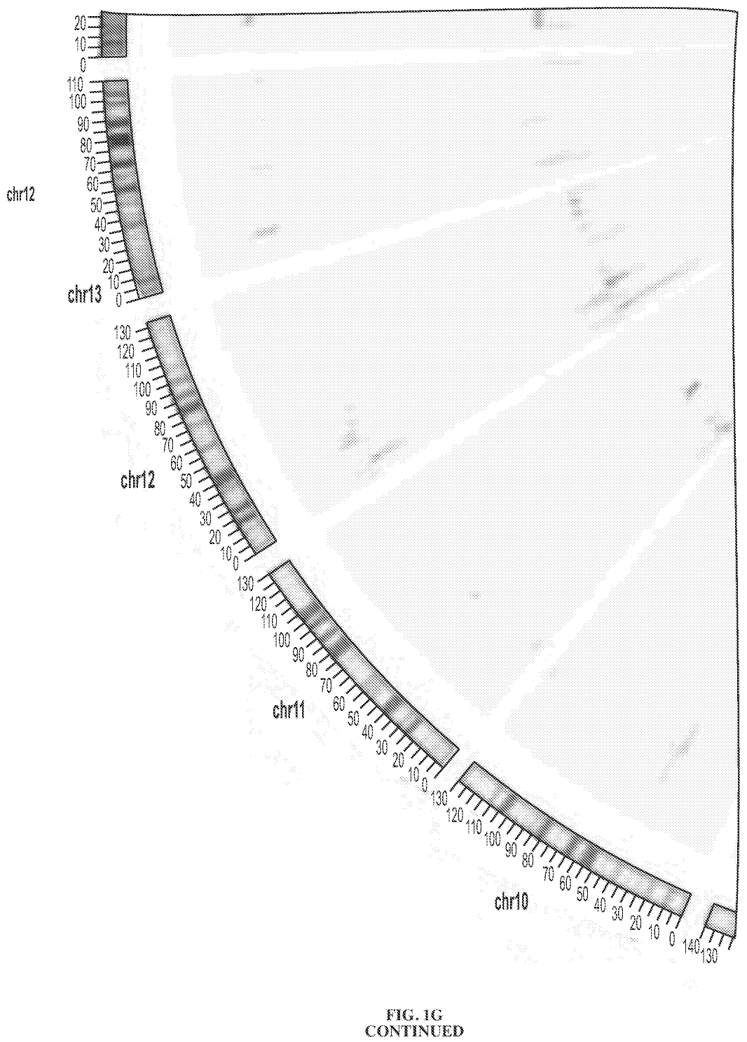

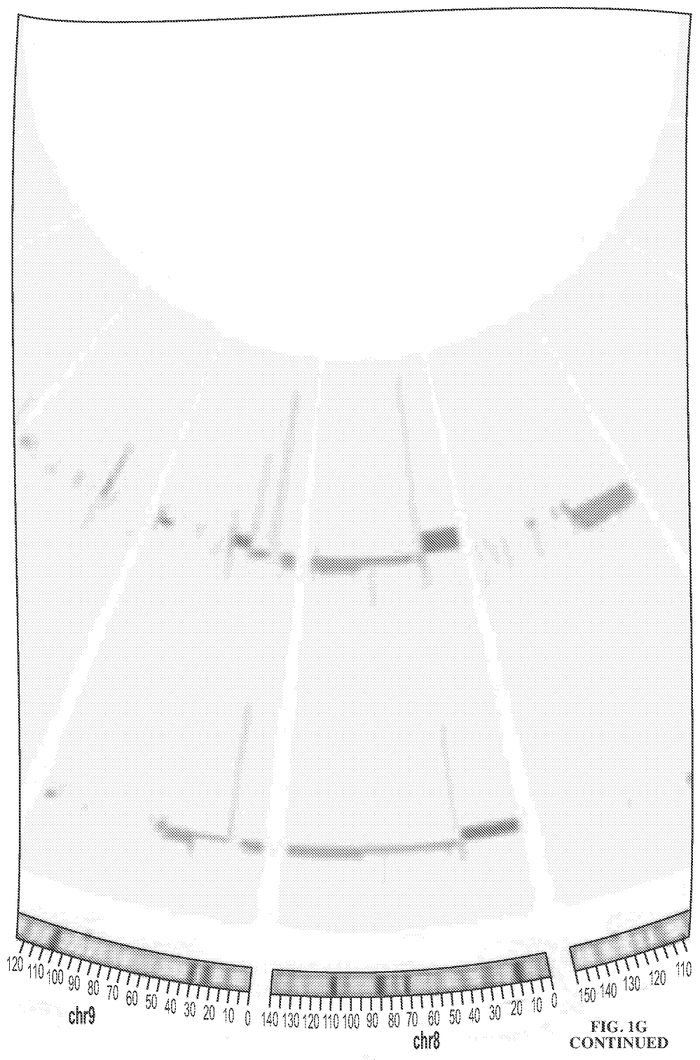

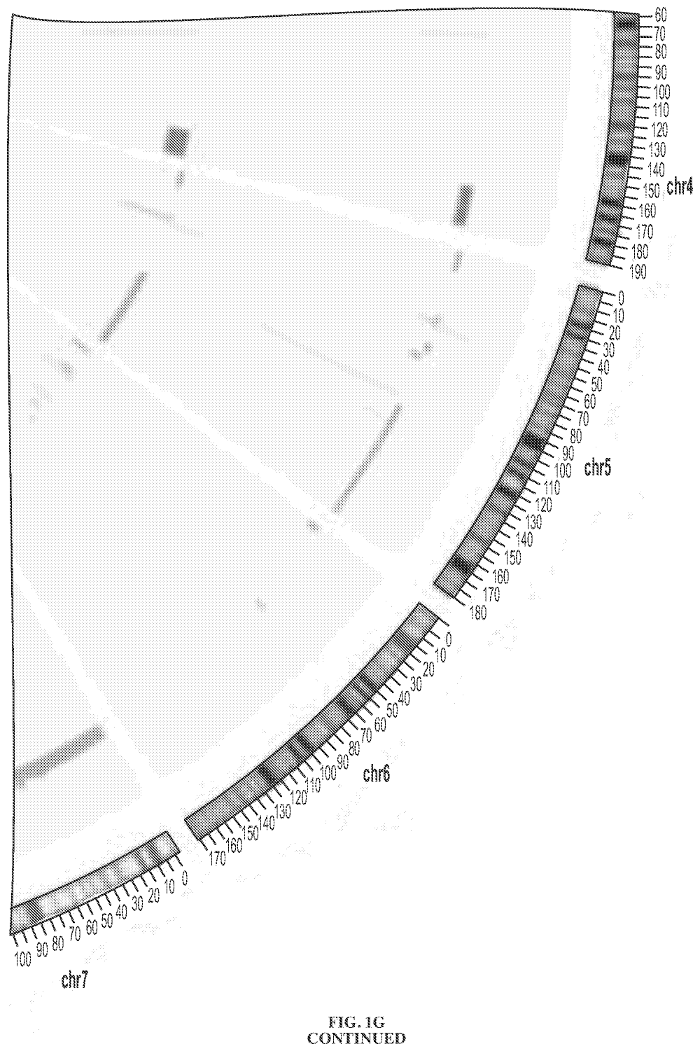

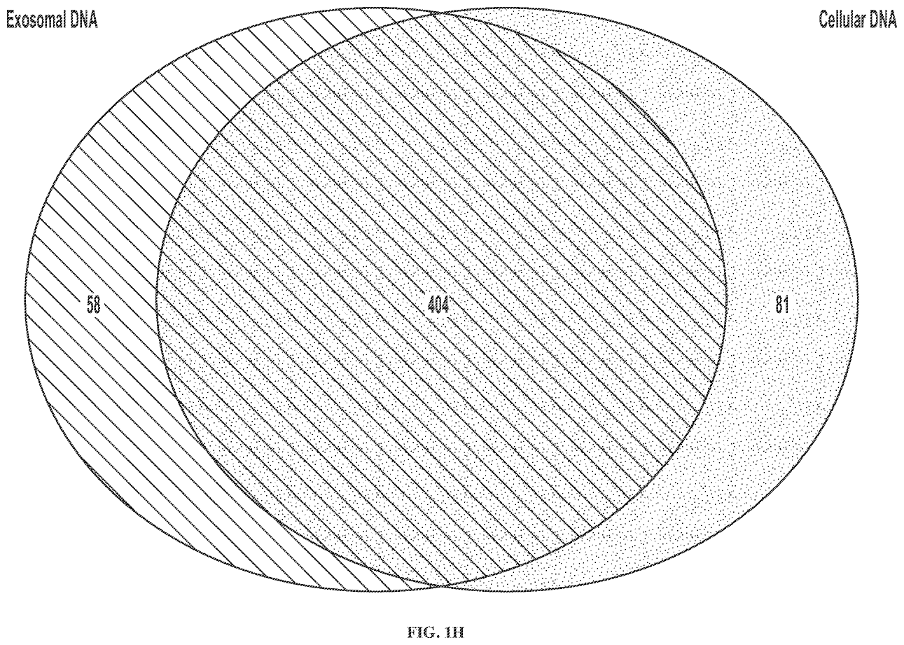

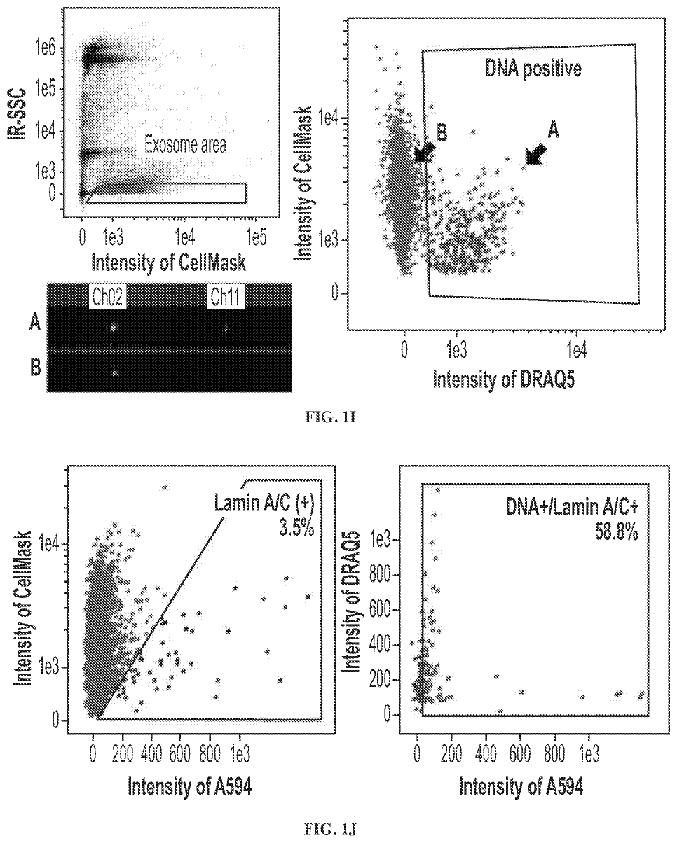

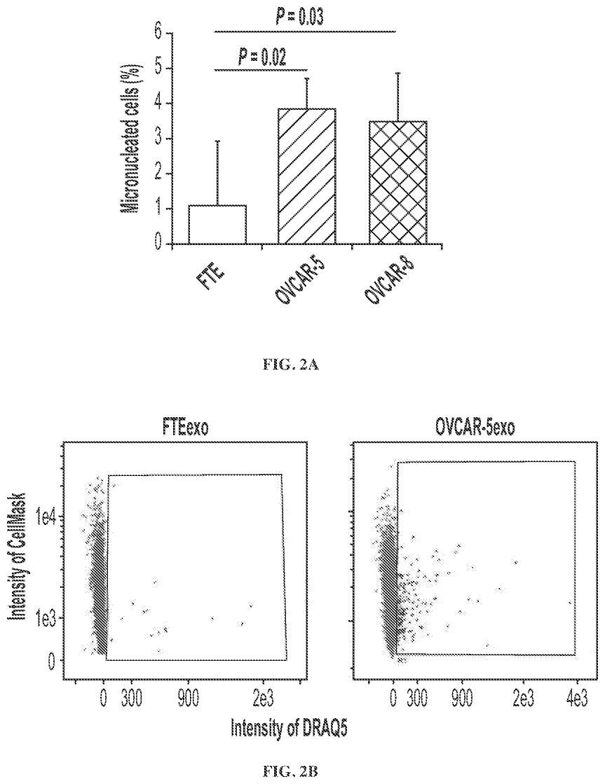

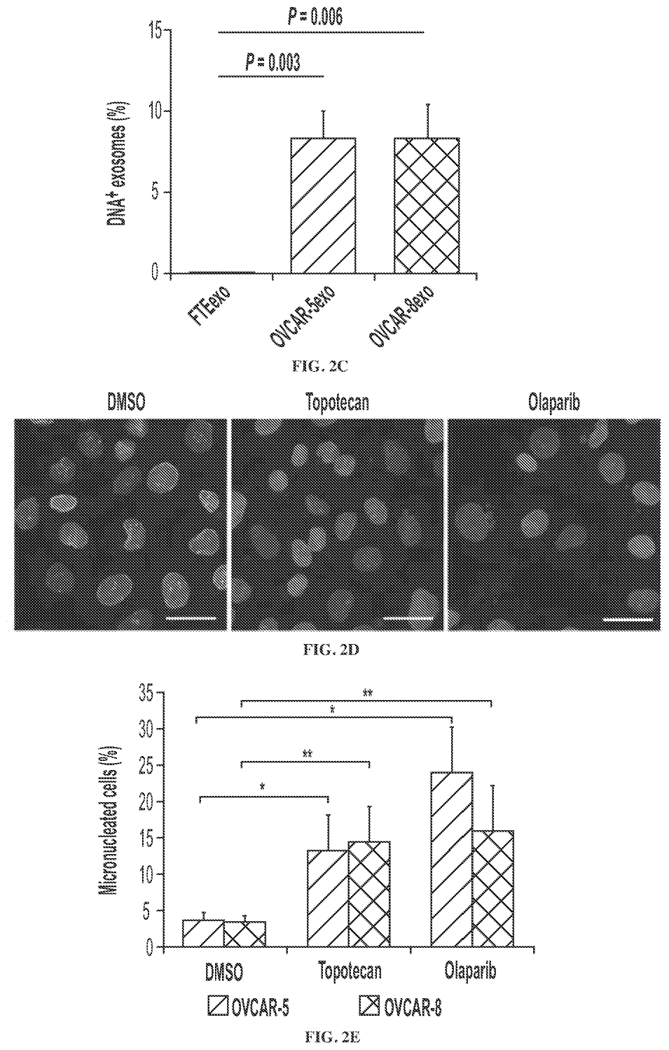

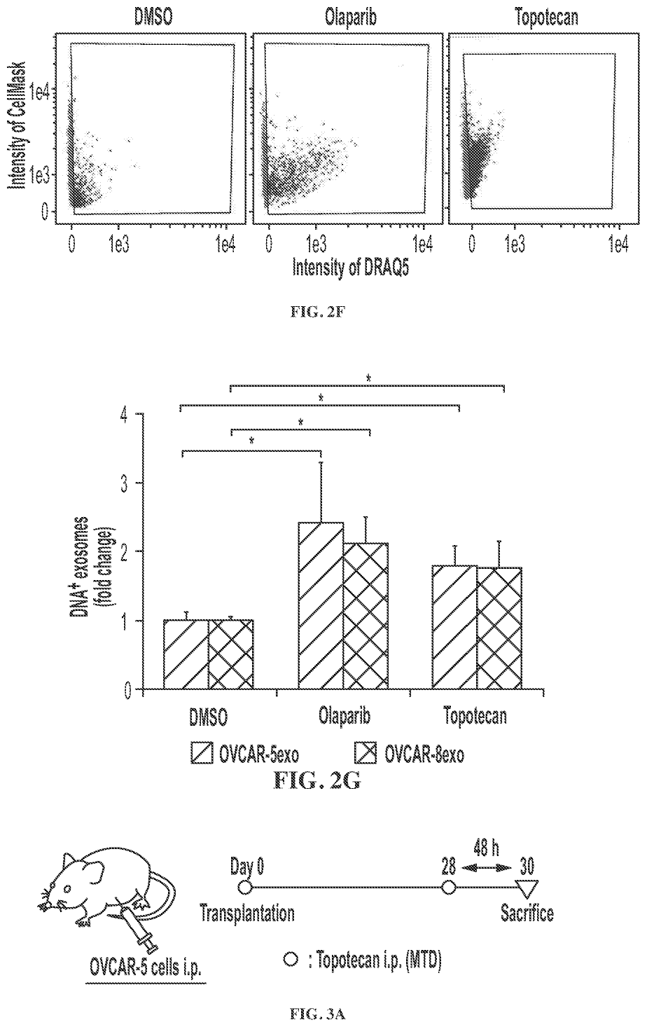

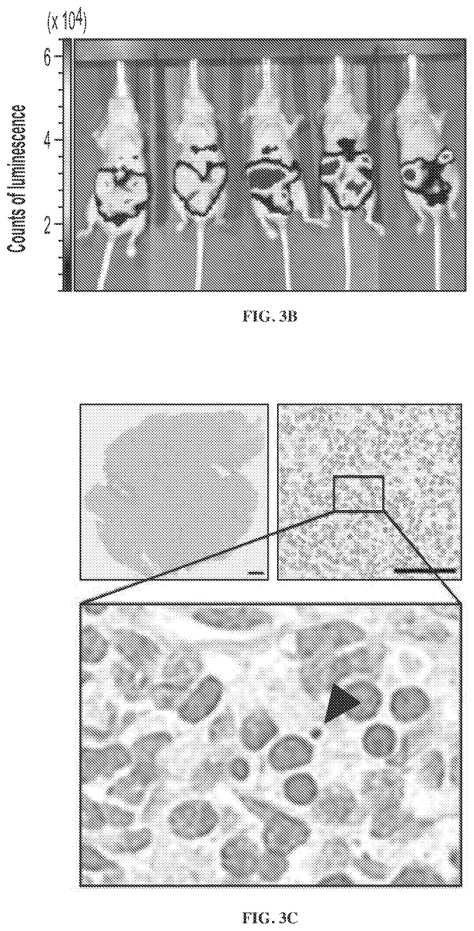

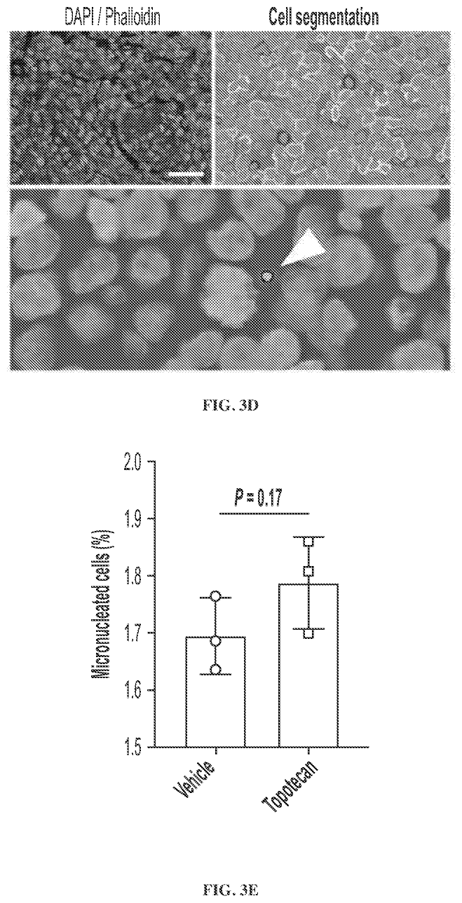

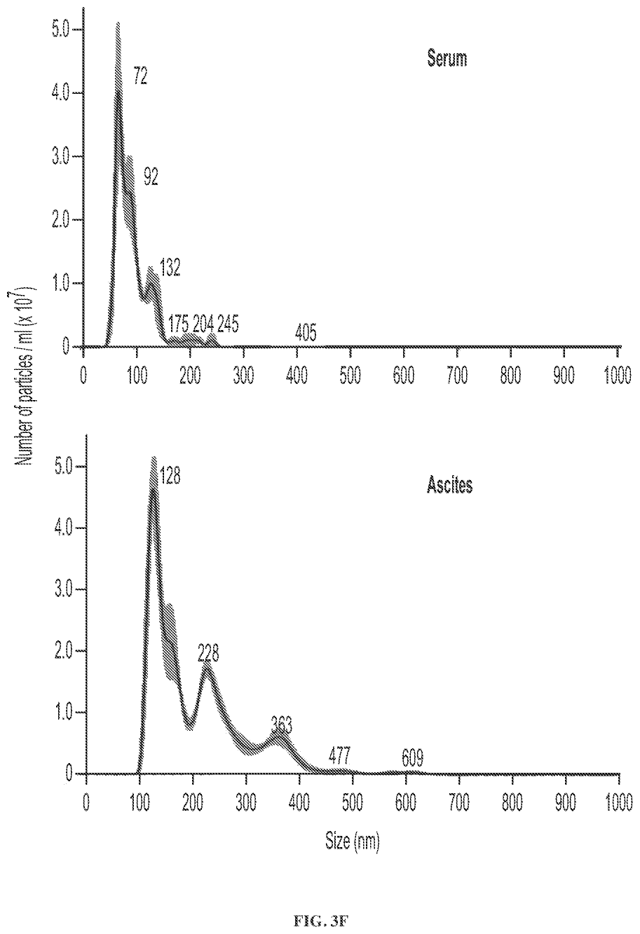

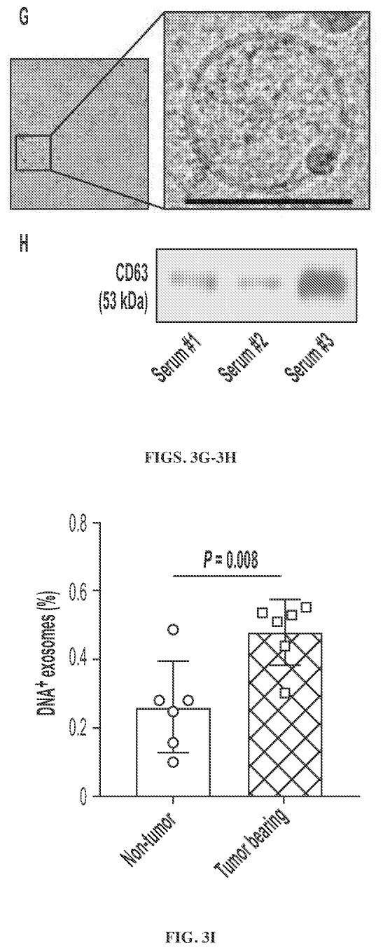

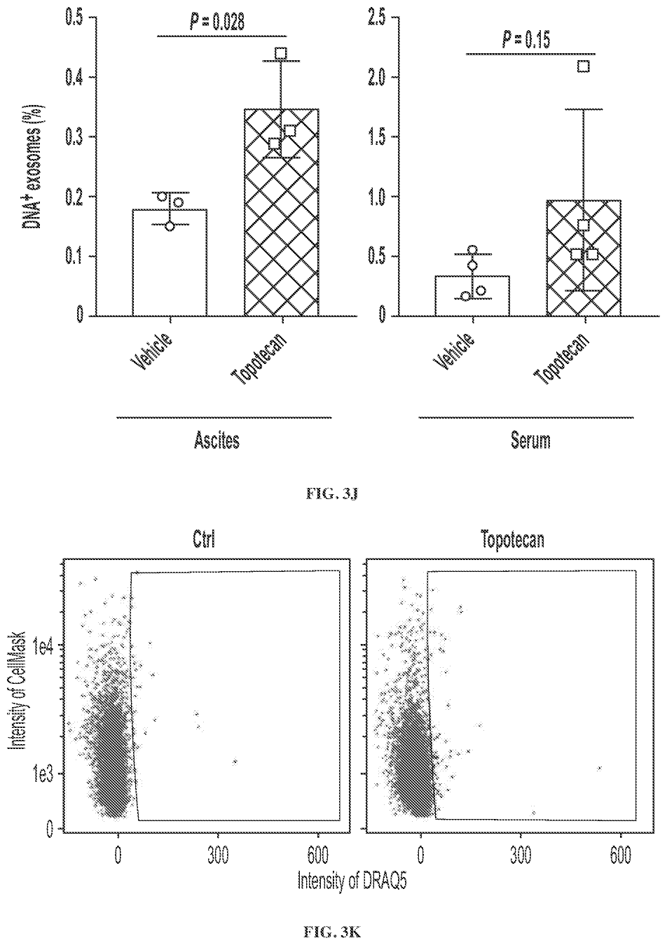

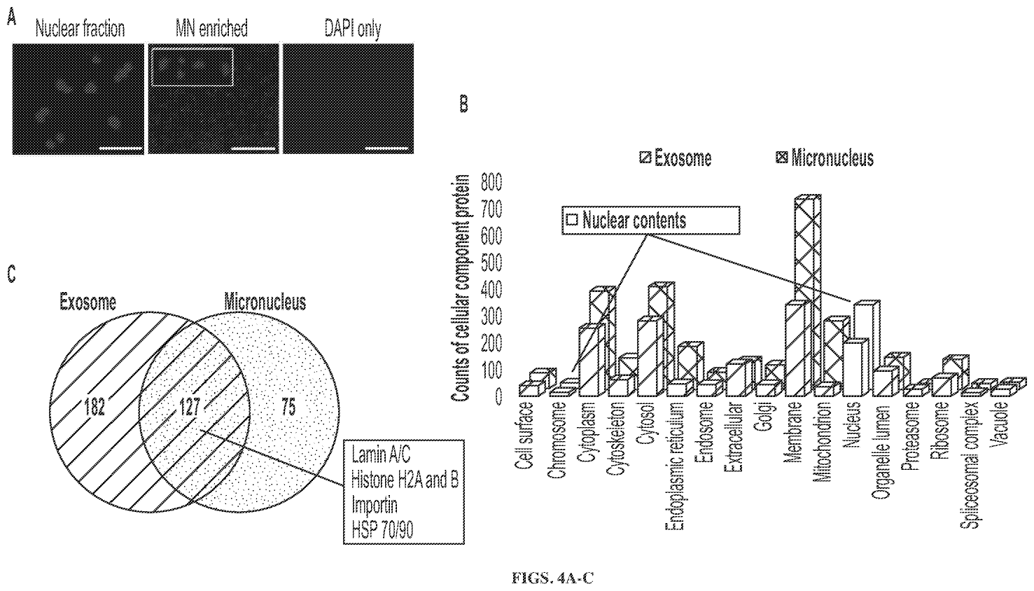

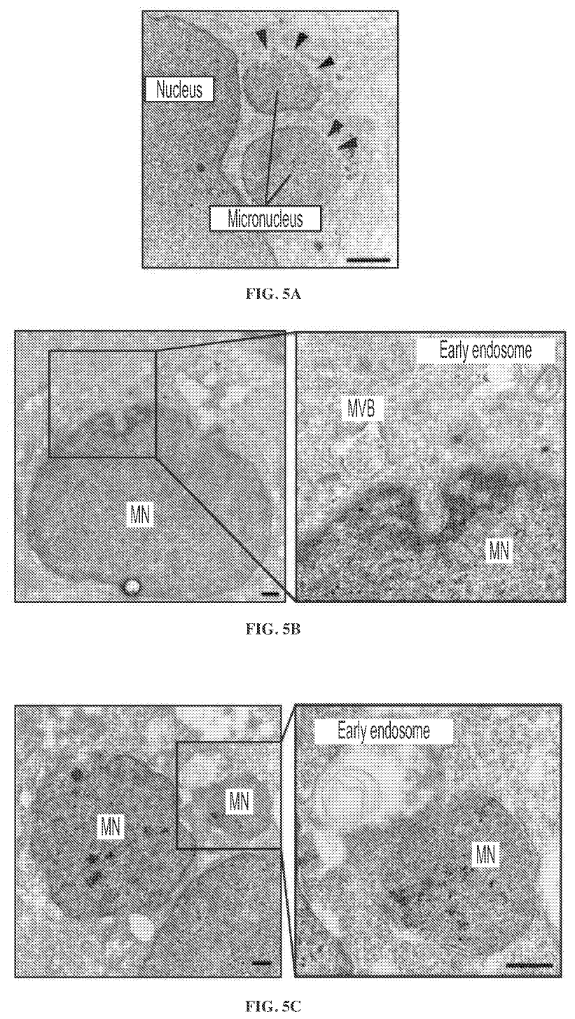

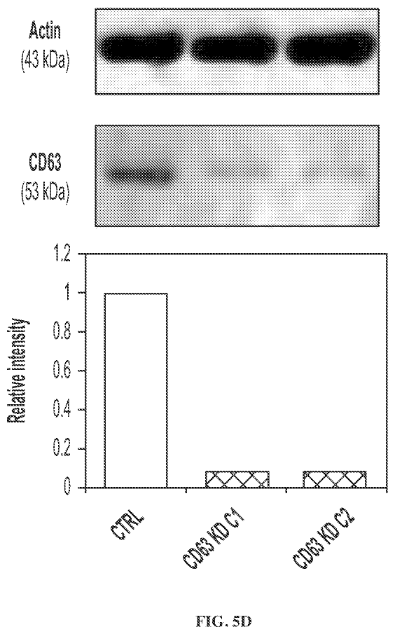

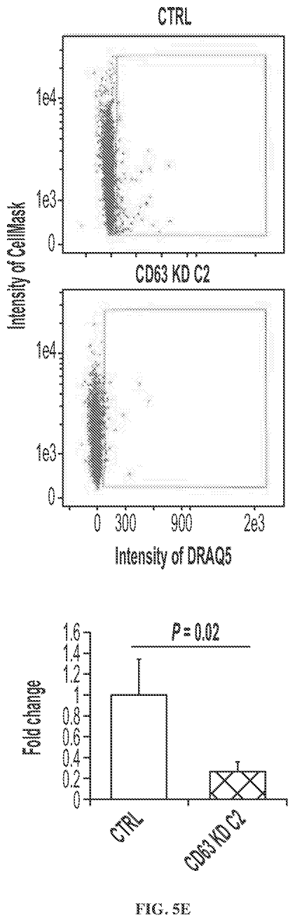

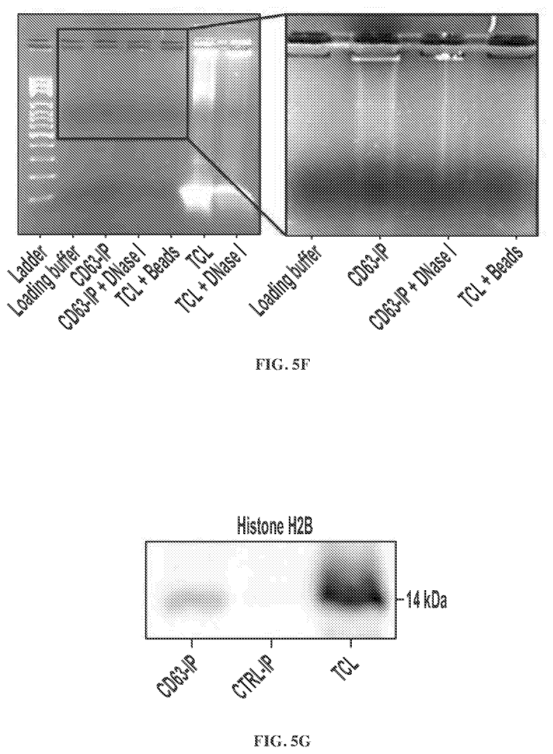

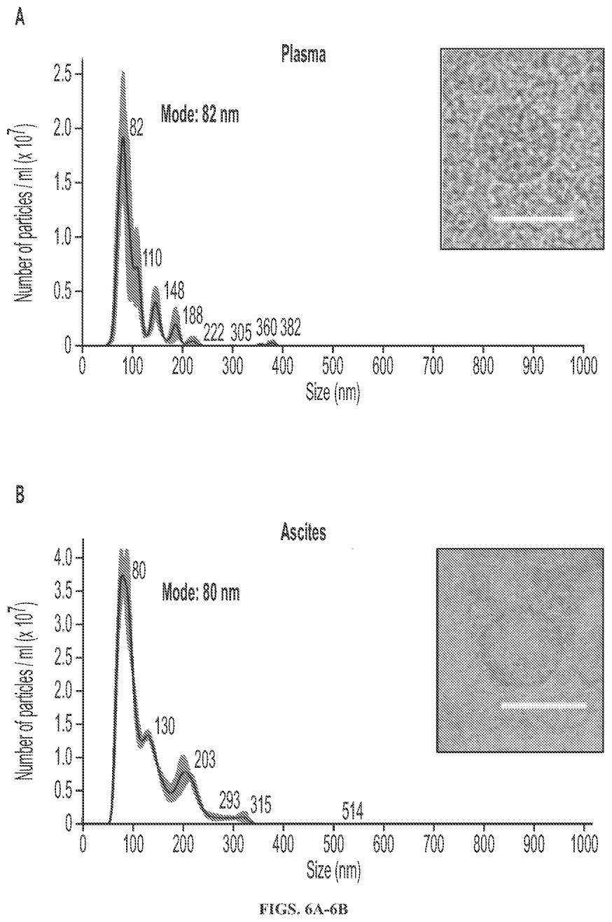

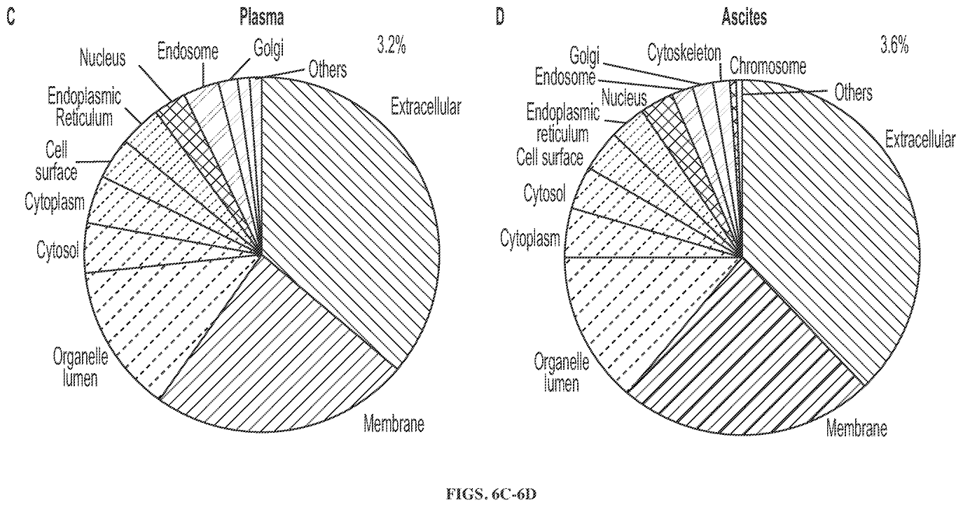

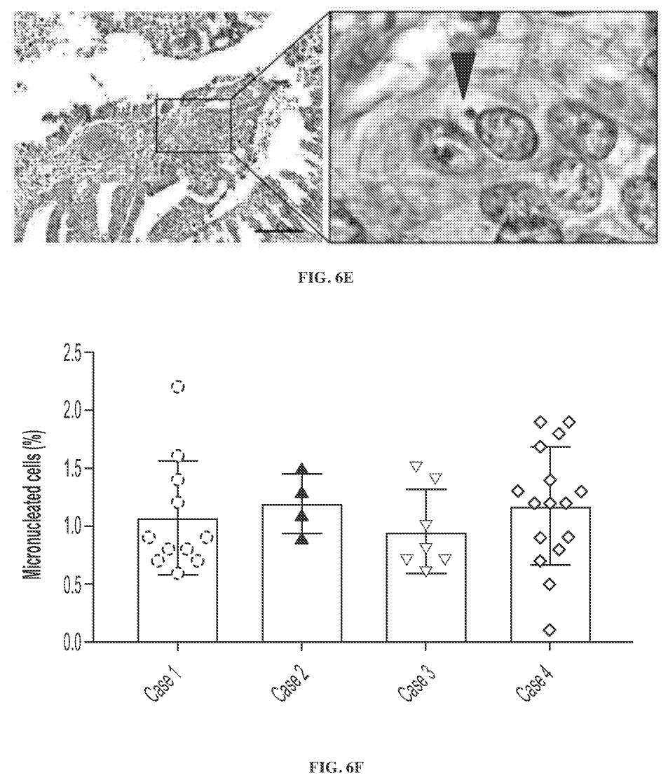

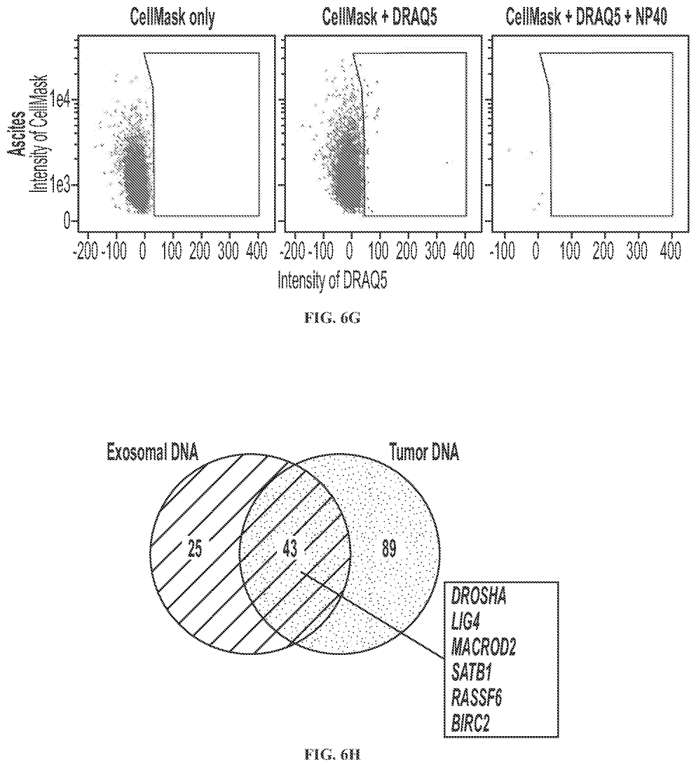

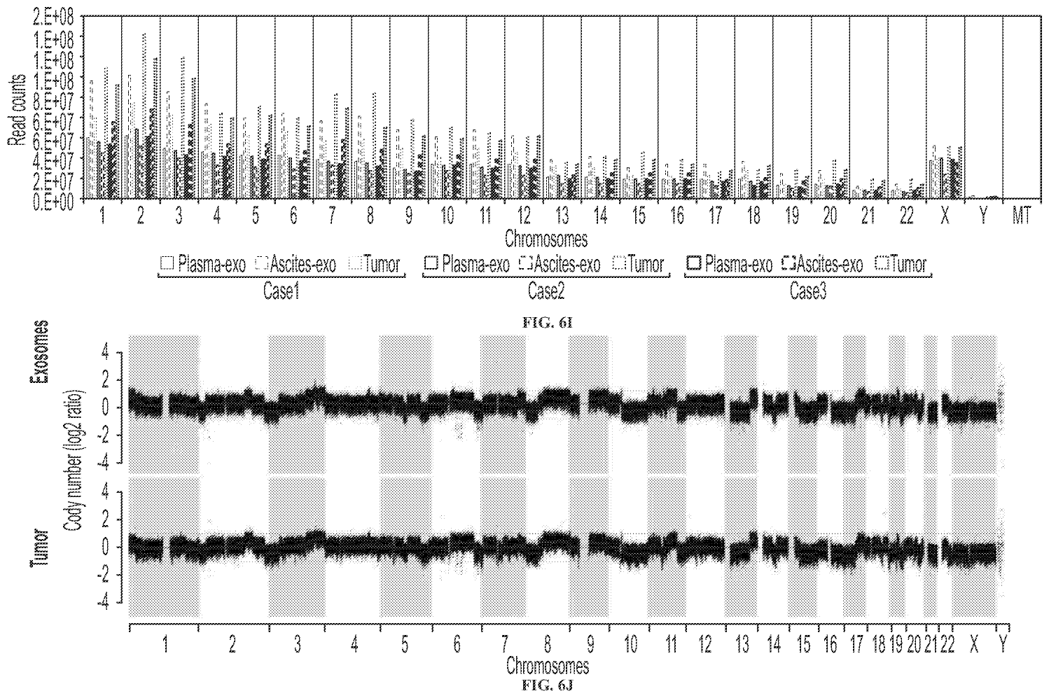

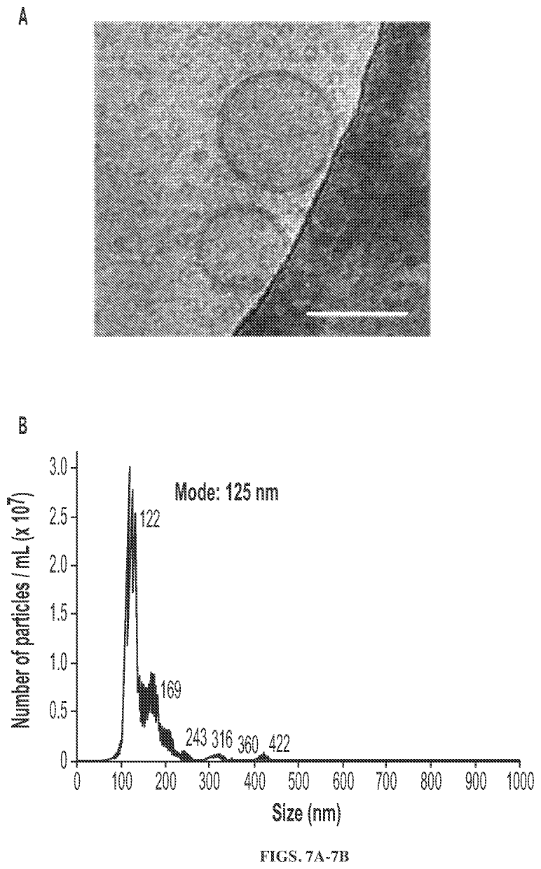

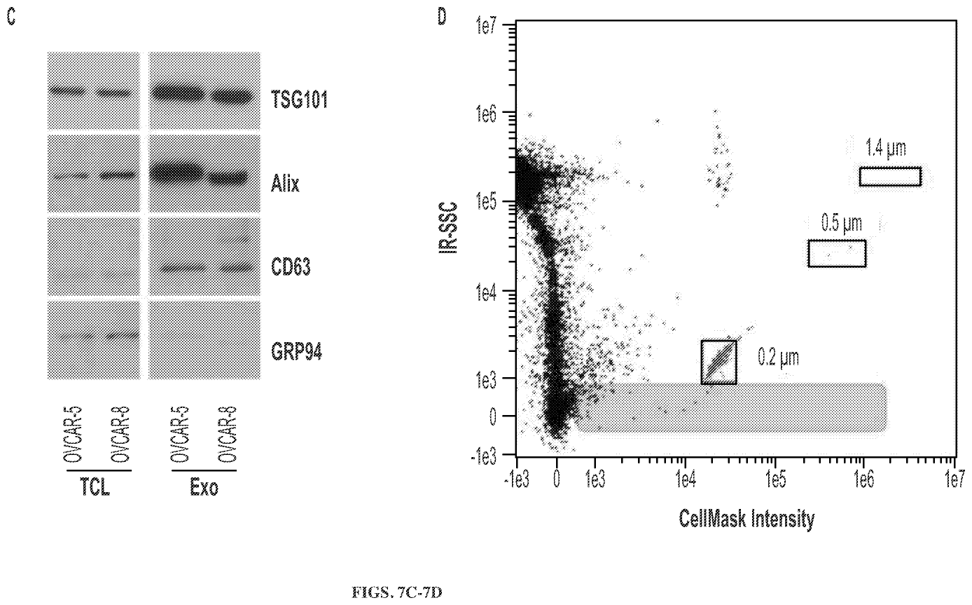

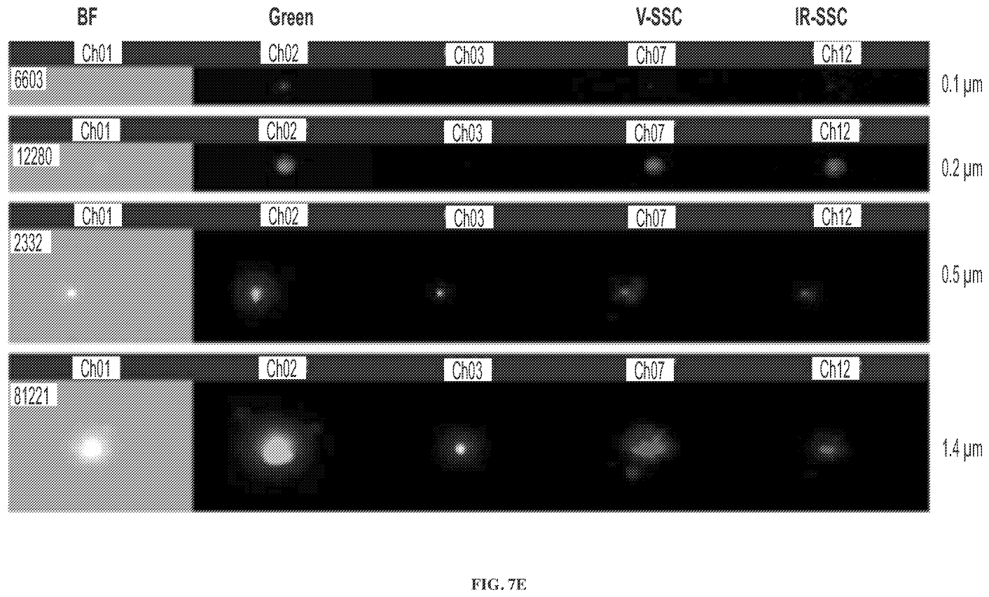

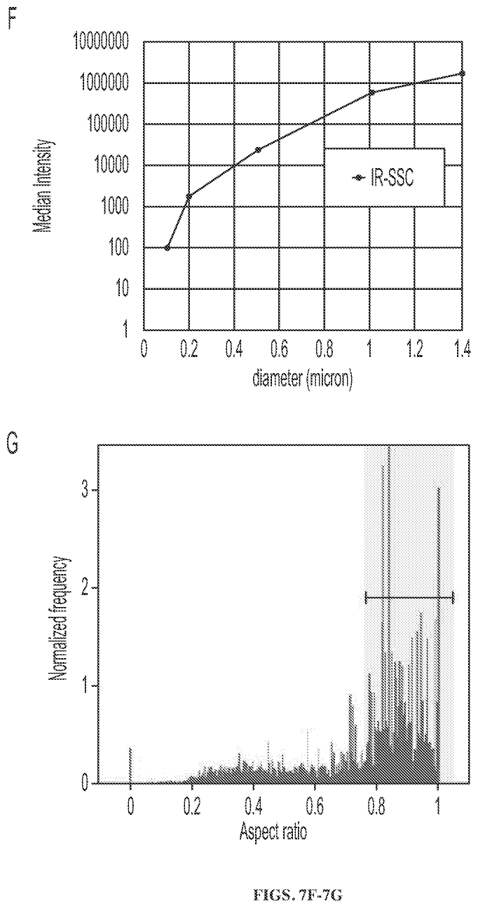

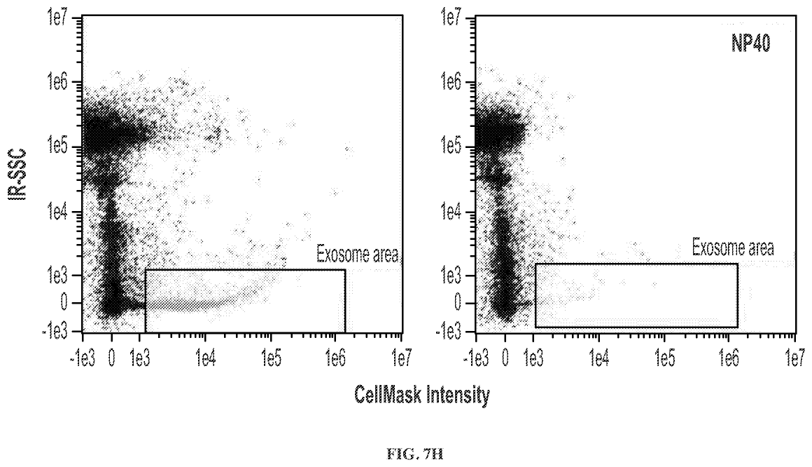

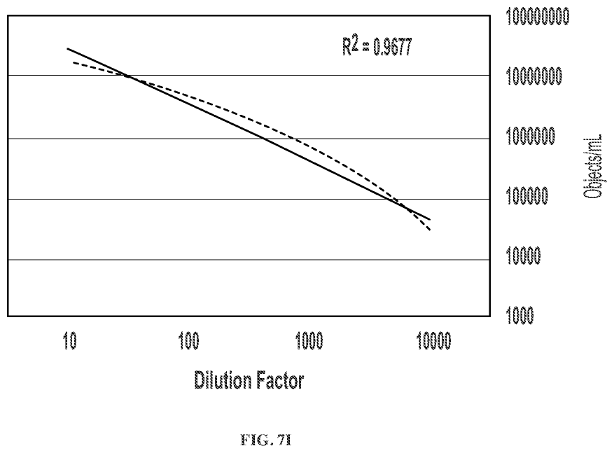

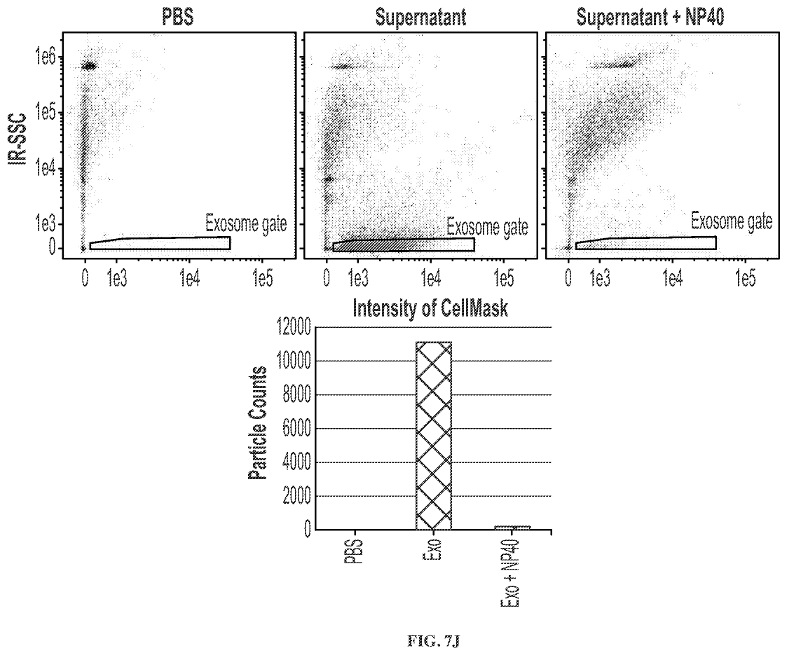

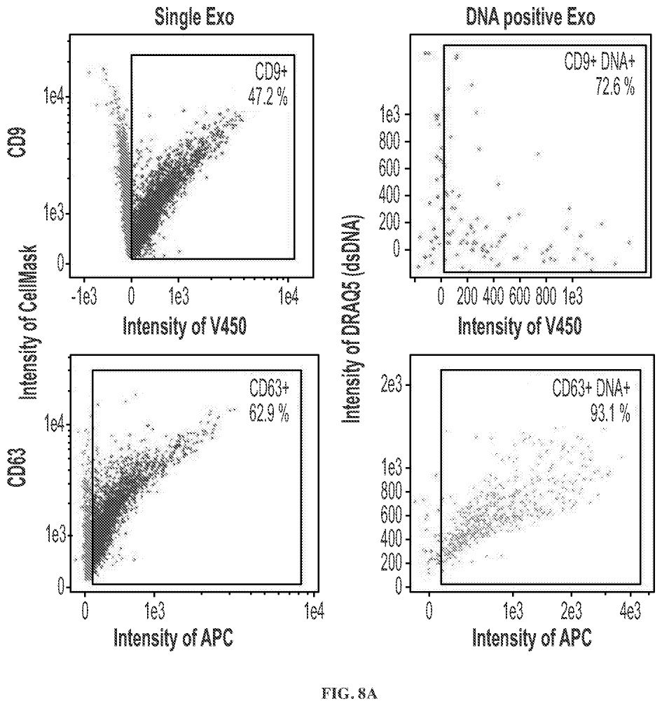

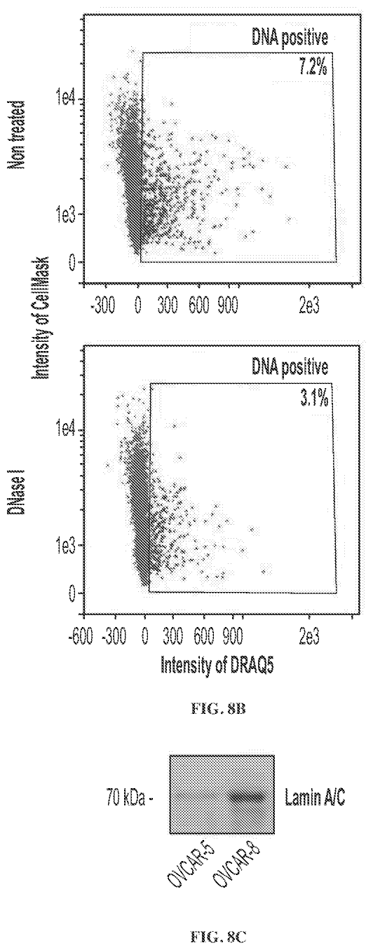

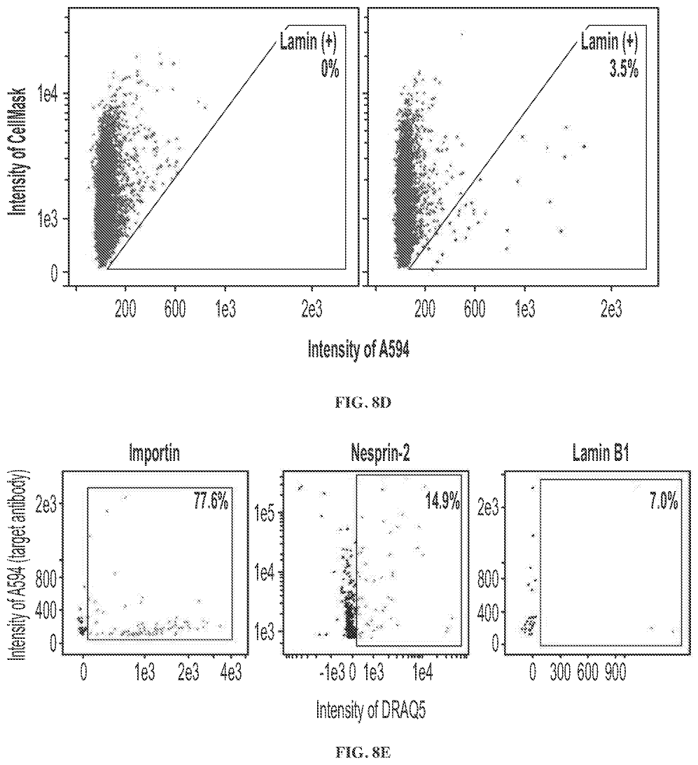

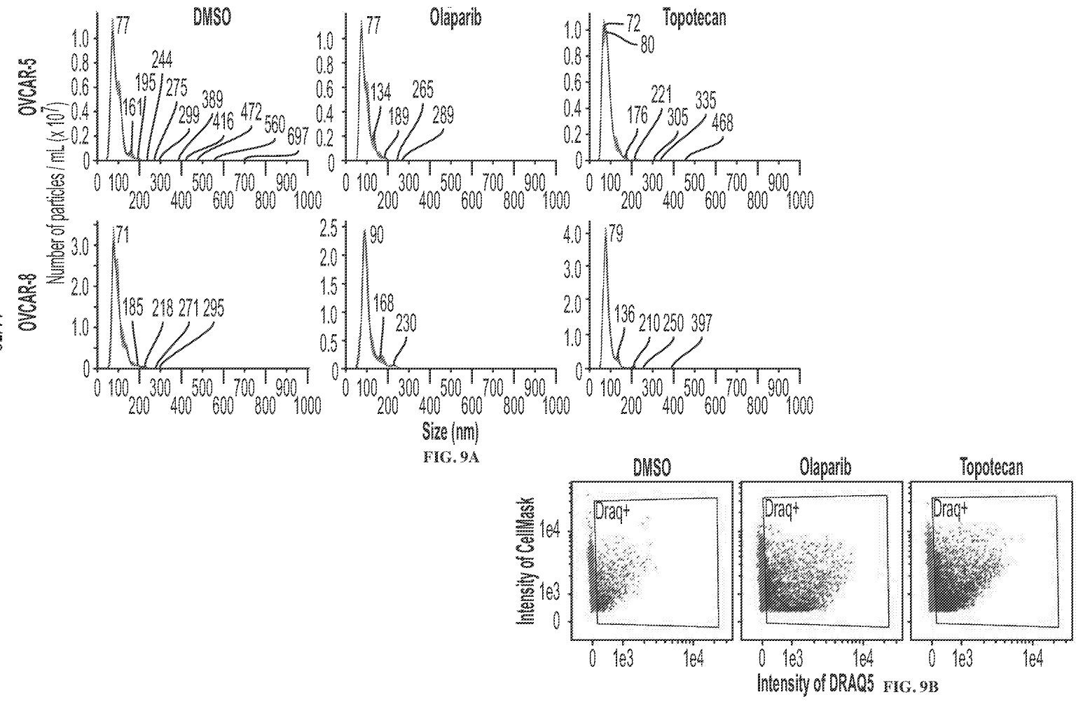

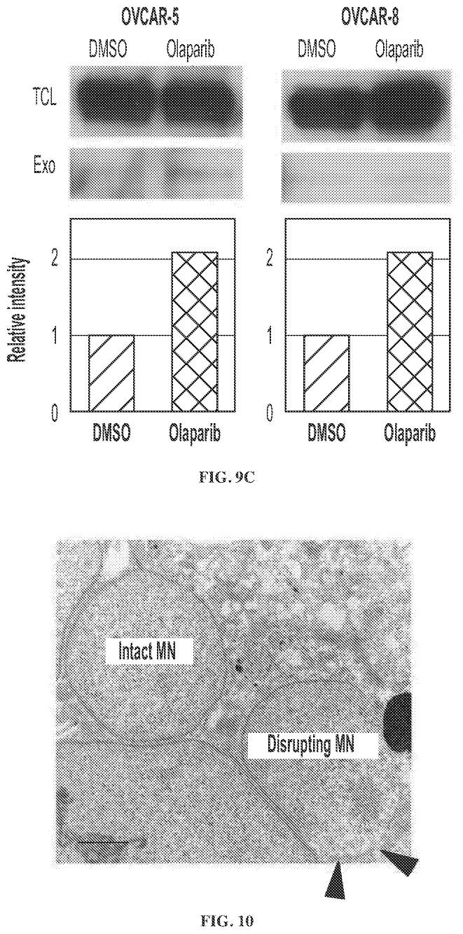

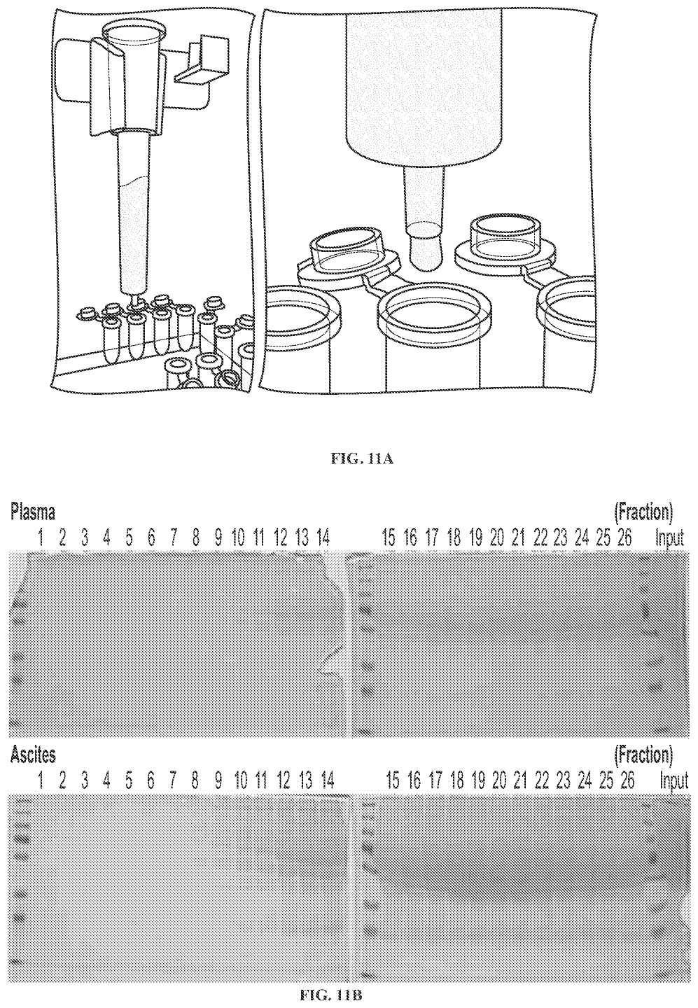

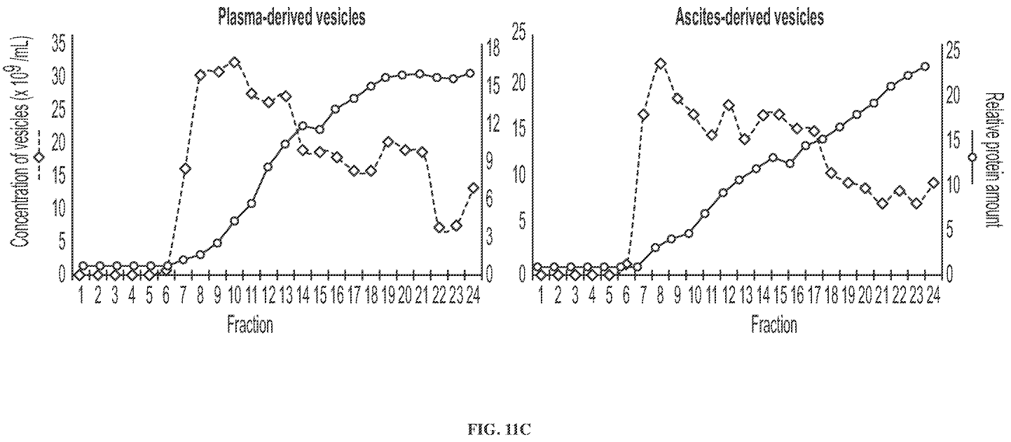

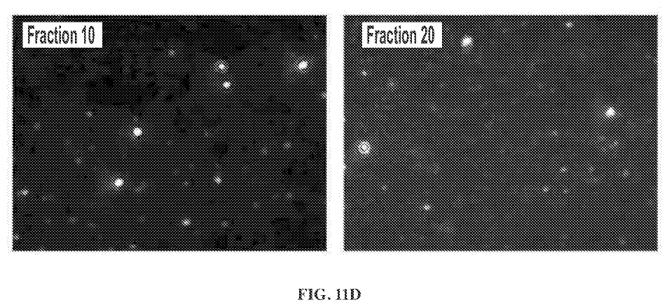

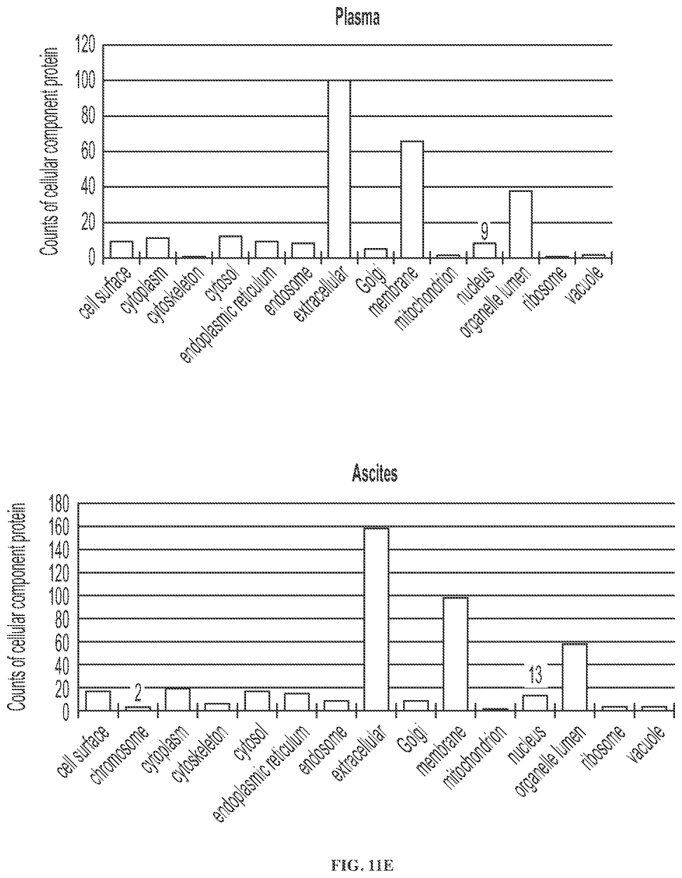

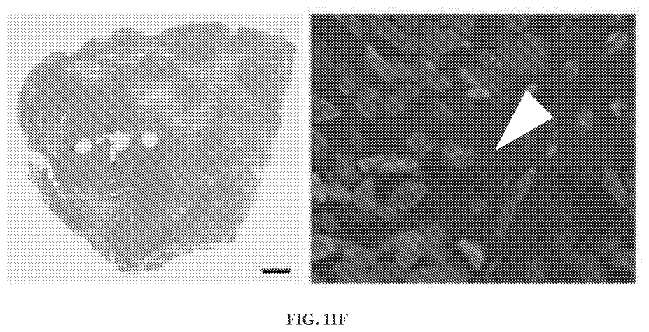

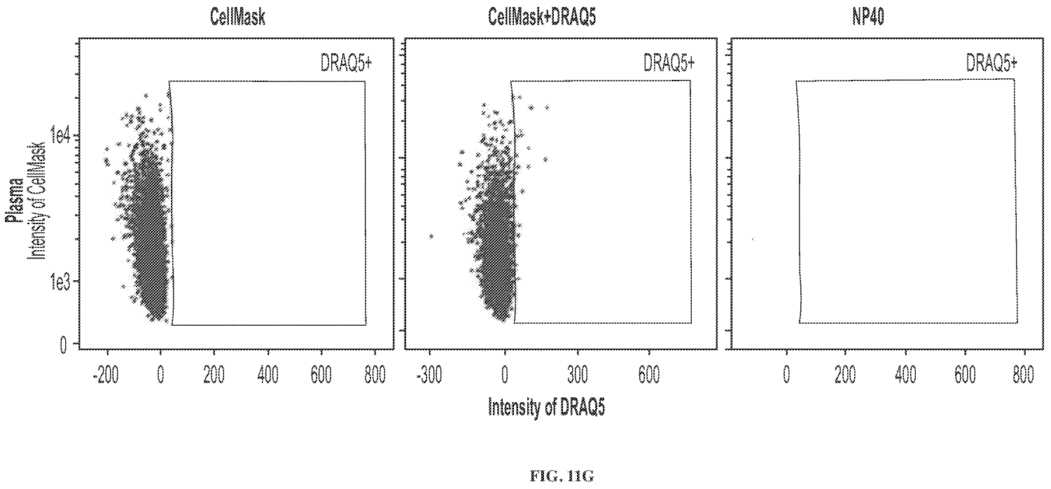

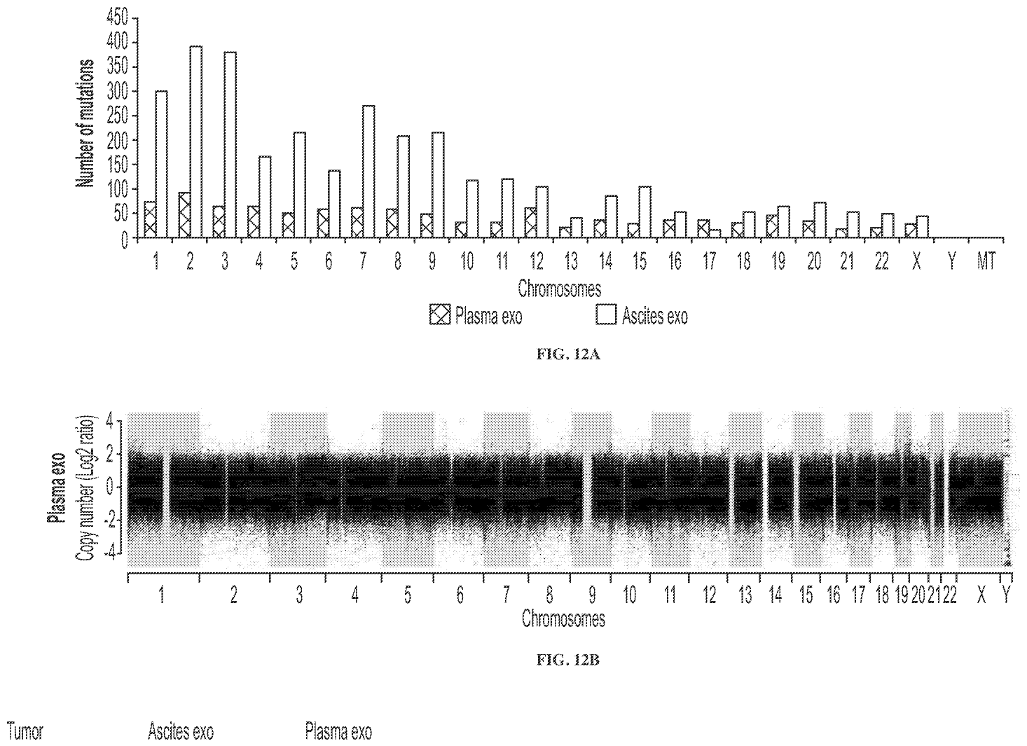

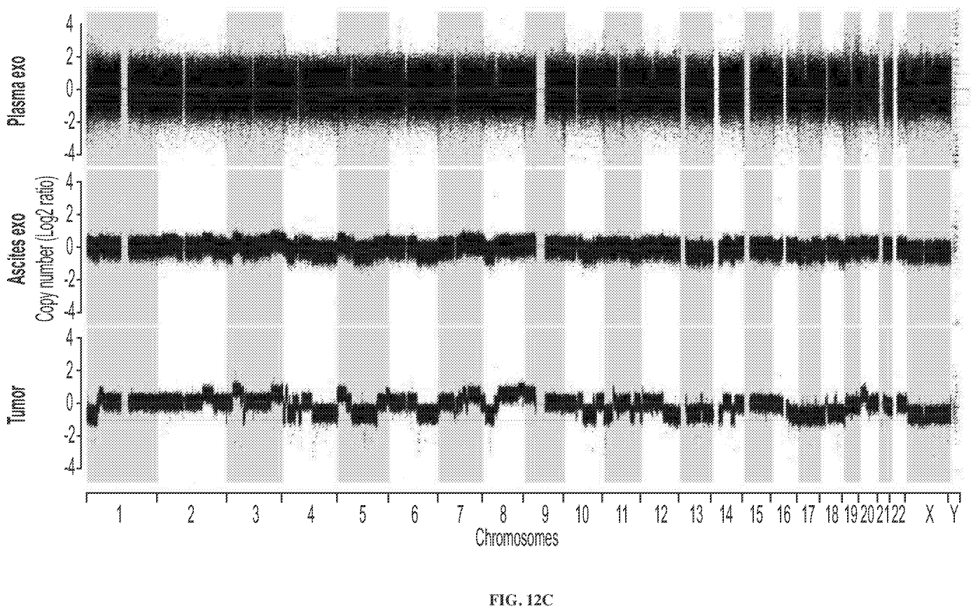

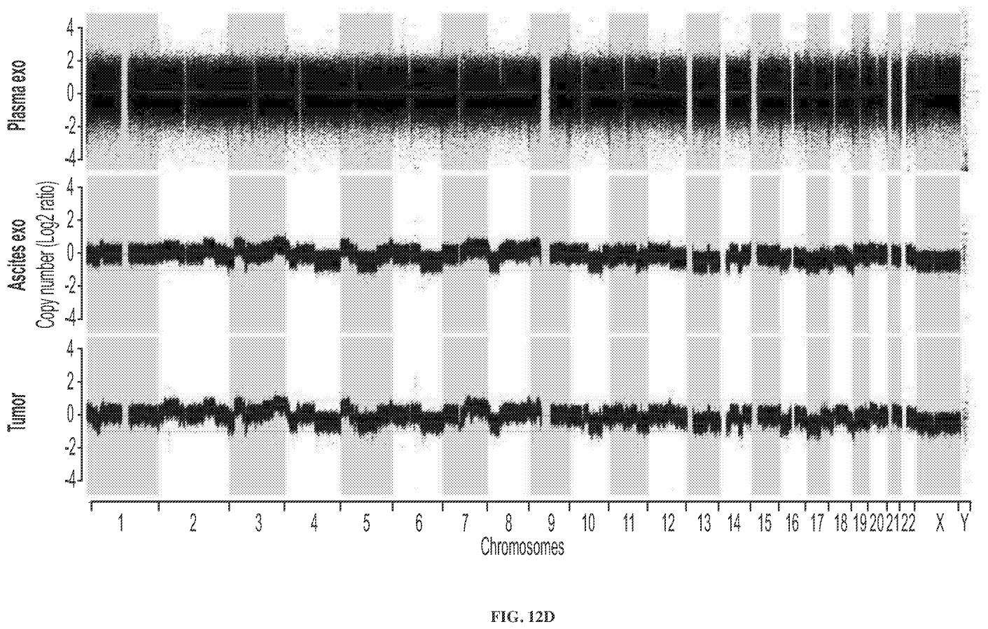

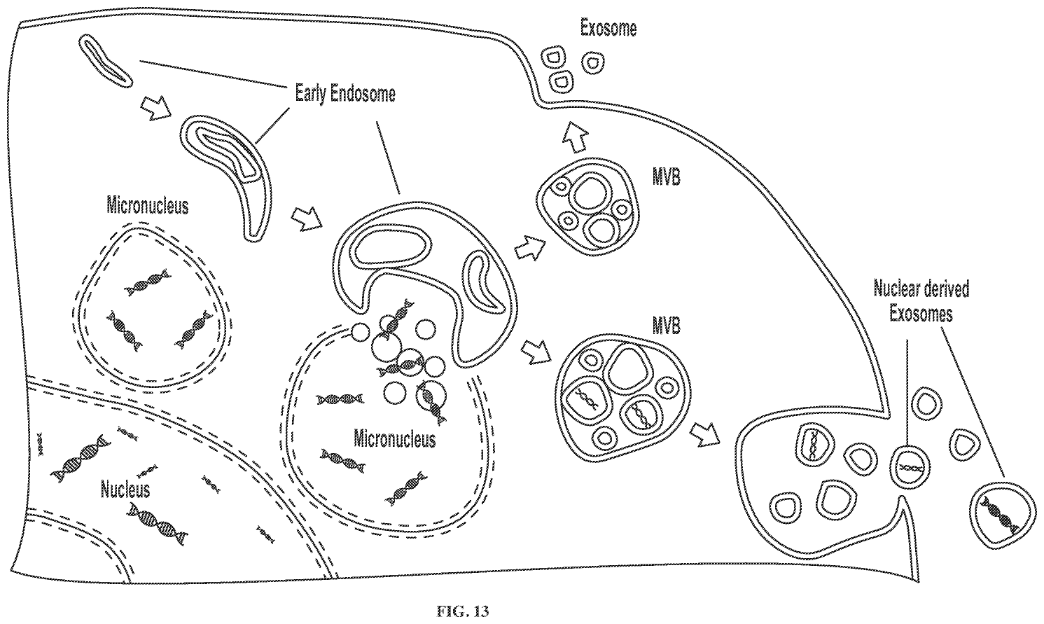

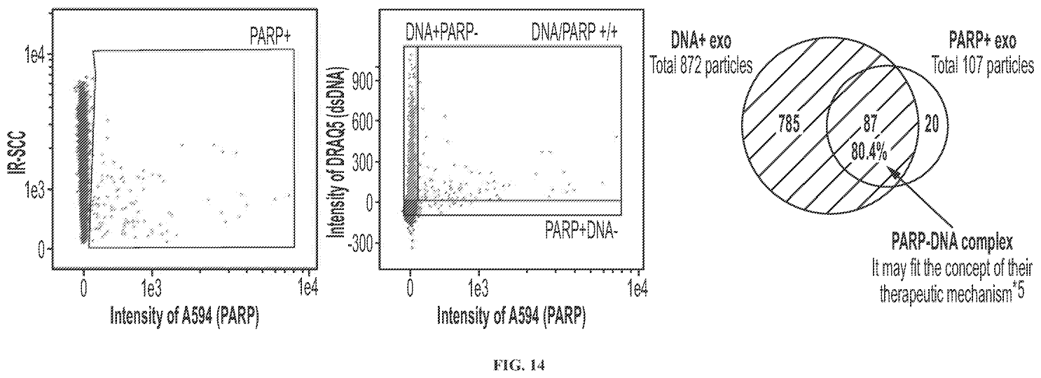

5, mitochondrion; 6, proteasome; 7, vacuole; 8, spliceosomal complex. (FIG. 1F) Counts of the cellular compartment origin of proteins resulting from MS analysis in OVCAR-5 cell—derived exosomes. The x axis represents the categories of cellular compartments. Nuclear proteins identified in chromosome and nucleus are highlighted. (FIG. 1G) CNVs of both the exosomal DNA (inner circle) and cellular DNA (outer circle), both derived from OVCAR-5 cells, are displayed on a chromosome map generated using Circos (v0.69.3). The outermost circle represents human chromosomes with coordinates (megabases). The histograms inside the inner circles represent copy number alterations identified by cnvkit. The larger the bar on the track, the larger the copy number alteration (log scale). (FIG. 1H) A Venn diagram of all the CNVs overlapping between the exosomal and cellular DNA derived from OVCAR-5 cells. (FIG. 11) Representative plots of OVCAR-5 exosomes from flow cytometry analysis. Top left: Particles are shown as black dots, and exosomes are in the boxed area. Right: Each dot indicates single exosomes stained with CellMask Green (Ch02), and the boxed area indicates DNA-positive particles stained with DRAQS (Ch11). Bottom left: Snapshots of individually stained exosomes. (A) and (B) are the exosomes present in the areas indicated in the right panel. (A) represents the DNA-positive exosomes, and (B) represents the negative exosomes. (FIG. 1J) Representative gate images of OVCAR-5 exosomes from imaging flow cytometry analysis. Left: Each gray dot indicates a single exosome, and the boxed area indicates a Lamin A/C—positive population. Right: All dots are from DNA-positive exosomes, and the boxed area indicates a Lamin A/C—positive population.

FIGS. 2A-2G. Promoting MN formation increases DNA-carrying exosomes. (FIG. 2A) Quantification of MN cells in FTE, OVCAR-5, and OVCAR-8 cells. MN counting is described in Materials and Methods. The experiment was performed in three independent biological replicates, and the average of the fold changes was calculated. Error bars are represented as SD. Statistical significance was determined by conducting an unpaired Student's t test. (FIG. 2B) Representative images of FTEexo and OVCAR-5exo from imaging flow cytometry analysis. The gates in both graphs indicate the DNA-positive population. (FIG. 2C) Population of DNA-positive exosomes in FTEexo, OVCAR-5exo, and OVCAR-8exo. The experiment was performed in three independent biological replicates, and the average of the fold changes was calculated. Error bars represent SD. Statistical significance was determined by conducting an unpaired Student's t test. (FIG. 2D) Representative images of nuclei from OVCAR-5 cells. Nuclei were IF-stained with Lamin A/C antibody. Scale bars, 50 μm. (FIG. 2E) Quantification of MN cells in OVCAR-5 and OVCAR-8 cells treated with DMSO, topotecan, and olaparib. The experiment was performed in three independent biological replicates, and the average of fold changes was calculated. Error bars represent SD. Statistical significance was determined by conducting an unpaired Student's t test. *P<0.05, **P<0.01. (FIG. 1F) Representative images of OVCAR-5exo in imaging flow cytometry analysis. (FIG. 1G) Population of DNA-positive exosomes in OVCAR-5exo and OVCAR-8exo. Parental cells were treated with DMSO, olaparib, or topotecan for 48 hours. FTEexo, OVCAR-5exo, and OVCAR-8exo indicate exosomes derived from FTE, OVCAR-5, and OVCAR-8 cells, respectively. The experiment was performed in three independent biological replicates, and the average of fold changes was calculated. Error bars represent SD. Statistical significance was determined by conducting an unpaired Student's t test. *P<0.05.

FIGS. 3A-3K. In vivo promotion of nExos with genotoxic drugs in ovarian cancer. (FIG. 3A) Schematic protocol for topotecan treatment. (FIG. 3B) Topotecan was administered intraperitoneally. All mice were euthanized on day 30. n of vehicle control-treated mice=4 and n of topotecan-treated mice=4. (FIG. 3C) Representative image of tumor tissue sections stained with hematoxylin and eosin (H&E). Scale bars, 700 μm (left panel) and 100 μm (middle panel). Black arrowhead indicates MN. (FIG. 3D) Left upper panel: Representative image of tumor tissue stained with DAPI and phalloidin. Scale bar, 50 μm. Right upper panel: Representative image of cell segmentation with Vectra imaging software. Lower panel: Representative image of the detected MN, indicated by white arrowhead. (FIG. 3E) Quantification of MN cells. MN counting is described in Materials and Methods. n of vehicle control-treated mice=3 and n of topotecan-treated mice=3. Error bars represent SD. Statistical significance was determined by conducting an unpaired Student's t test. (FIG. 3F) Nanoparticle tracking analyses for exosomes derived from plasma and ascites. (FIG. 3G) Representative cryo-EM images of ascites exosomes from OVCAR-5 intraperitoneal model. Scale bar, 100 nm. (FIG. 3H) Western blot of mouse plasma exosomes (n=3). (FIG. 31) Population of DNA-positive exosomes in serum from non-tumor-bearing mouse and OVCAR-5 intraperitoneal model. n=each 6. Error bars represent SD. Statistical significance was determined by conducting unpaired Student's t test. (FIG. 3J) Population of DNA-positive exosomes in serum and ascites from the mice treated with topotecan or vehicle. n=ascites, each 3 and serum, each 4. Error bars represent SD. Statistical significance was determined by conducting unpaired Student's t test. (FIG. 3K) Representative images of serum exosome in imaging flow cytometry analysis.

FIGS. 4A-4C. MN and nExo contain similar protein content. (FIG. 4A) Representative images of nuclear fraction and MN-enriched fraction. All samples were obtained from OVCAR-5 cells and stained with DAPI. The white box in the middle image is a magnified view. Scale bar, 50 μm. (FIG. 4B) Counts of protein cellular compartment of origin resulting from MS analysis in exosome and MN-enriched fraction. Samples were obtained from OVCAR-5 cells. The x-axis represents the categories of cell components. Proteins from nuclear and chromosome compartments highlighted in yellow. (FIG. 4C) Venn diagram showing overlapping proteins between exosome and MN-enriched fraction. Samples were obtained from OVCAR-5 cells. Lamin A/C, Histone H2A/B, importin, and heat shock protein (HSP) 70/90 were included in the overlapped 127 proteins.

FIGS. 5A-5G. Cargo of disrupted MN is loaded into nExos. (FIGS. 5A-5C) Representative images of TEM of OVCAR-5 cells. Black arrowheads indicate the disrupted nuclear envelop of MN. Scale bars, 2 μm (FIG. 5A) and 500 nm (FIG. 5B and FIG. 5C). (FIG. 5D) Western blot of OVCAR-5 cells with CD63 knockdown (CD63 KD). CTRL indicates the OVCAR-5 cells transfected with a scramble shRNA sequence. Densitometry analysis is quantified in the bar chart. (FIG. 5E) Representative images of exosome from CD63-knockdown OVCAR-5 cells in imaging flow cytometry analysis. The fold change of nExo population. The experiment was performed in three independent biological replicates, and the average of fold changes was calculated. Error bars represent SD. Statistical significance was determined by conducting an unpaired Student's t test. (FIG. 5F) The samples of OVCAR-5 cells with IP experiments for CD63 were analyzed with 2% agarose gel electrophoresis, and DNAs were visualized by ethidium bromide staining. (FIG. 5G) Western blot of OVCAR-5 cells with IP experiments for CD63. CTRL-IP indicates the OVCAR-5 cells treated with negative control immunoglobulin G (IgG) in the Universal Magnetic Co-IP Kit (54002, Active Motif).

FIGS. 6A-6J. Detection of nExo in clinical samples. (FIG. 6A and 6B) NTA for exosomes was derived from plasma and ascites from patients with high-grade serous ovarian carcinoma, along with representative cryo-EM images. Scale bars, 100 nm. (FIG. 6C and 6D) Pie charts of the cellular compartment of origin of proteins based on MS analysis from plasma- and ascites-derived exosomes. Nuclear components are highlighted in red. (FIG. 6C) “Others” includes cytoskeleton, mitochondrion, ribosome, and vacuole. (FIG. 6D) “Others” includes mitochondrion, nucleus, organelle lumen, ribosome, and vacuole. (FIG. 6E) Representative tissue image of high-grade serous ovarian carcinoma stained with H&E. Black arrowhead indicates MN. Scale bars, 100 μm. (FIG. 6F) Population of micronucleated cells in high-grade serous ovarian carcinoma tissue. Pretreated tissue slides were obtained and analyzed as described in Materials and Methods. Each dot indicates one view for analysis. n=4. (FIG. 6G) Representative imaging flow cytometry images of exosomes obtained from ascites of patients with high-grade serous ovarian carcinoma. (FIG. 6H) Venn diagram showing overlapping exonic mutated genes between tumor and ascites exosomes. Samples were obtained from patients with HGSC. DROSHA, LIG4, MACROD2, SATB1, RAS SF6, and BIRC2 were included in the 43 overlapping genes. (FIG. 6I) Read counts of all chromosomes in plasma- and ascites-derived exosomal DNA and tumor DNA. All three cases were from patients with advanced-stage HGSC. (FIG. 6J) CNV status in gDNA in ascites-derived exosomal DNA and corresponding primary tumor from patients with HGSC. Profiles demonstrate somatic chromosomal gains (upper) and losses (lower), as well as normal polymorphisms.

FIGS. 7A-7J. Optimization of imaging flow cytometry analyses for exosomes. (FIG. 7A) Cryo-electron microscopic image of exosomes isolated from OVCAR-8 cells. Scale bars, 100 nm. (FIG. 7B) Nanoparticle tracking analysis for exosomes isolated from OVCAR-8 cells. (FIG. 7C) Western blot of exosome markers in OVCAR-8. TSG101, Alix, and CD63 are used as exosome markers and GRP94 as a cellular marker. TCL, total cell lysate; Exo, exosomes. (FIG. 7D) Scatter plot of sidescatter (Side-SCatter; SSC) intensity versus CellMask intensity resolving for four discrete populations (different bead sizes: 1.4, 0.5, and 0.2 μm). The 1.4- and 0.5-μm beads were loaded separately, and the plotted gates were indicated as squares. The predicted area, which indicates exosome population, is shown as a shaded area. (FIG. 7E) Images for bright field (BF), CellMask, and IR-SSC. (FIG. 7F) Plot for particle size standard. Vertical axis indicates median intensity of beads; horizontal axis indicates diameter. (FIG. 7G) Plot for gating SSC intensity aspect ratios over 0.8. (FIG. 7H) Scatter plots for exosomes. Exosomes were derived from HI0180 cells, which are non-transformed human ovarian surface epithelial cells. The boxed areas indicate exosome areas. Right panel indicates exosomes after addition of NP40. (FIG. 7I) Dilution factor for exosomes by imaging flow cytometry analyses. Pearson's Correlation value was calculated. (FIG. 7J) Scatter plots for OVCAR-5 cell culture supernatant. The supernatants were spun down at 500 ×g for 10 min and 2,000 ×g for 30 min. The resulting supernatant was then filtered through a 0.2-μm filter. Exosomes were stained by CellMask as described in the Methods section. The number of exosomes is shown in the bar chart.

FIGS. 8A-8E. Characterization of nExo. (FIG. 8A) Representative images of OVCAR-5 exosomes from imaging flow cytometry analysis, using gating analysis shown in FIG. 7H. Left panel: boxed areas indicate CD9 or CD63-positive population. Right panel: boxed areas indicate CD9 or CD63/DNA double-positive exosomes. (FIG. 8B) Scatter plots for OVCAR-5 exosomes treated with DNase I. The exosomes were stained by CellMask Green and DRAQS. Samples were treated with DNase I as described in the Materials and Methods section. (FIG. 8C) Western blot of Lamin A/C in exosomes. (FIG. 8D) Representative images of OVCAR-5 exosomes from imaging flow cytometry analysis. Left panel: no antibody was added. Right panel: Lamin A/C antibodies were added. (FIG. 8E) Representative images of OVCAR-5 exosomes from imaging flow cytometry analysis. Importin, Nesprin-2, and Lamin B1 antibodies were added to exosome samples.

FIGS. 9A-9C. Analyses of cell MN and exosomes treated with genotoxic drugs. (FIG. 9A) Nanoparticle tracking analyses for exosomes derived from cells treated with genotoxic drugs. (FIG. 9B) Representative scatter plots of exosomes derived from OVCAR-8 cells treated with genotoxic drugs in imaging flow cytometry analysis. (FIG. 9C) Western blots of exosomes derived from cells treated with olaparib. Densitometry analysis is quantified in the bar chart. TCL, total cell lysate; Exo, exosomes.

FIG. 10. Imaging of MN in ovarian cancer cells. Representative images of TEM of OVCAR-5 cells. Black arrowheads indicate the disrupted nuclear envelope of micronuclei. MN, micronucleus; Scale bars, 2 μm.

FIGS. 11A-11G. Characterization of nExo and MN in human samples. (FIGS. 11A-11D) Size Exclusion Chromatography for Exosome Isolation. (FIG. 11A) Photos for procedure on fractionation. (FIG. 11B) Representative images of SDS PAGE gel stained by SimplyBlue™ SafeStain (LC6060; Invitrogen). (FIG. 11C) Plots for concentration of vesicles and protein amount. (FIG. 11D) Representative image of nanoparticle tracking analysis for fractions 10 and 20 from patient plasma. Clearness of background was different in both samples. (FIG. 11E) Counts of the cellular compartment origin of proteins resulting from MS analysis in human plasma and ascites-derived exosomes. X-axis represents the categories of cell compartments. Nuclear proteins identified in chromosome and nucleus are highlighted. (FIG. 11F) Left panel: representative image of human tumor tissue sections stained with hematoxylin and eosin (H&E). Scale bars, 2 mm. Right panel: representative image of tumor tissue stained by DAPI. Representative image of the detected micronuclei by Vectra imaging software, indicated by white arrowhead. (FIG. 11G) Representative images of exosome from high-grade serous ovarian carcinoma patients' plasma in imaging flow cytometry analysis.

FIGS. 12A-12D. WGS for clinical samples. (FIG. 12A) Mutation calls across all chromosomes and mitochondrial DNA. Data is generated by using plasma and ascites derived exosomal DNA from high-grade serous carcinoma (HGSC) patients. Over 20 reads of mutated genes were defined as detected mutation. (FIGS. 12B-12D) CNV status in genomic DNA in plasma and ascites derived, exosomal DNA and corresponding primary tumor of HGSC patients. All 3 cases were from advanced stage ovarian HGSC patients. Case 1 (A) is from same patients in FIG. 6J. Profiles demonstrate somatic chromosomal gains (upper) and losses (lower), as well as normal polymorphism.

FIG. 13. Proposed model for the mechanism underlying nExo synthesis. Collapse of micronuclei in the cell cytoplasm serve as a source of nuclear content being shuttled into multivesicular bodies (MVBs) via tetraspanins and secreted in exosomes.

FIG. 14. Colocalization of PARP-positive exosomes with gDNA.

### DETAILED DESCRIPTION

Provided herein are analyses of nExo isolated from ovarian cancer cells and characterizations to explore potential clinical relevance such as cancer detection and therapeutic response. Nuclear derived exosomes (nExo) are predominantly secreted from cancer cells and could serve as an important biomarker. Although the subpopulation is relatively rare, their presence in bio-fluids isolated from cancer patients holds the potential of serving as novel biomarkers for early detection and therapeutic response to genotoxic agents.

Exosomes are small extracellular vesicles that mediate biological and cellular functions including cell-to-cell communication (Thery et al., 2002). Exosomes are generated in early endosomes and then stored in multivesicular bodies (MVBs) that fuse with the plasma membrane and release the exosomes to the extracellular space (Raposo and Stoorvogel, 2013). Tetraspanins, proteins known as major exosomal markers, that localize to endosomes, MVBs, and exosomes are thought to be responsible for trafficking cargo into these organelles (Pol and Klumperman, 2009). The ability of exosomes to induce distinct biological behaviors in either the cells that secrete them or the recipient cells depends on their cargo (Valadi et al., 2007). Exosome cargoes are variable and can include proteins and RNA species (Mateescu et al., 2017). The presence and abundance of protein and RNA, including microRNAs, can influence cell behavior in several contexts, ranging from immune system activation to suppression of solid tumor growth and cancer metastasis (Kosaka et al., 2016).

Recent studies have demonstrated the presence of genomic DNA (gDNA) and nuclear proteins within exosomes (Balaj et al., 2011). The presence of gDNA in exosomes has been associated with processes such as cell senescence and stimulation of the cGAS/STING inflammatory pathway (Takahashi et al., 2017). In addition, gDNA is predominantly detected in exosomes derived from cancer cells rather than healthy cells. Paradoxically, gDNA is mainly confined to the cell nucleus and does not normally interact with the cytoplasmic MVBs that give rise to exosomes (Thakur et al., 2014). Thus, the mechanisms by which nuclear components are present in exosomes remain poorly understood.

One possibility involves micronuclei (MN), which are cytoplasmic structures enveloped by a nuclear membrane and are generated when the cell nucleus fails to properly segregate nuclear material, including chromosomes. (Fenech et al., 2011). This mis-segregation can arise during mitosis and can be driven by DNA-damaging agents, such as radiation, which can result in chromosome fragmentation (Crasta et al., 2012). The presence of MN serves as a surrogate marker of genomic instability, which is a hallmark of cancer (French, 2007). The nuclear envelope that surrounds MN is highly unstable, eventually breaking down and exposing its contents to the cytoplasm during cell division (Hatch et al., 2013). Induction of MN formation can promote activation of the cGAS/STING pathway, resulting in an inflammatory response against both senescent and cancer cells (Harding et al., 2017).

Herein, a previously unidentified interaction between MN and nExo is identified. Induction of MN formation using genotoxic drugs promotes nExo release. These results suggest a mechanism for the origin of gDNA in exosomes whereby, following MN collapse, their nuclear contents are shuttled into MVBs via tetraspanins. Moreover, exosomal gDNAs in ovarian cancer patients reflect copy number variation (CNV) status of the primary tumor, revealing informative DNA mutations. Thus, nExos can serve as important biomarkers for cancer detection and longitudinal monitoring of cancer patients.

### I. ASPECTS OF THE PRESENT DISCLOSURE

Herein, a subpopulation of nExos is identified and characterized, and mechanistic insights into the packaging of their nuclear content is provided. The imaging flow cytometry—based method allowed accurate quantification of the nExo subpopulation both in vivo and in vitro. A link between MN formation and the generation of nExos upon induction of genomic instability with genotoxic drugs was identified. In addition, MN collapse serves as a source of nuclear content shuttled into MVB via tetraspanins (FIG. 13). Last, the presence of MN and nExos was identified in the ascites of patients with ovarian cancer, which holds promise for rapid assessment of the genomic status of those tumors.

As described above, cancer cells secrete more nExo than normal cells. However, <1% of nExo from human plasma and ascites samples contained gDNA, and this ratio was consistent with the in vivo mouse model data. On the basis of the finding that nontransformed cells release fewer nExos, this discrepancy between the proportions of in vitro and in vivo DNA-containing exosomes (around 10% and less than 1%, respectively) is not surprising; tumor-derived exosomes in biofluids are likely a subset of a much larger population of non-tumor cell-derived exosomes. Given the higher secretion of nExo by cancer cells, the presence and quantity of nExos could serve as a cancer biomarker. In addition, the gDNA in nExos can be interrogated for tumor-associated genetic alterations.

Genotoxic drugs induce MN formation and nExo release. Herein, topotecan and olaparib were used because of their direct effects on DNA repair mechanisms (Creemers et al., 1994; Bitler et al., 2017). Although both drugs have different mechanisms for targeting cancer cells, both can induce genomic instability and induce apoptosis. MN serve as a marker of genomic instability, and they form either by chromosome mis-segregation during mitosis or from direct insults to gDNA (Jagetia and Reddy, 2002). The in vivo mouse experiments revealed a trend toward an increased number of micronucleated cells in the topotecan-treated group, but this difference was not statistically significant, which may reflect the single dose of topotecan used for this experiment. In addition, a significant increase in the concentration of nExos was observed in the ascites of tumor-bearing mice, but not in the serum. This difference may be due to an insufficient dose of the drugs, a small sample size, or a larger fraction of tumor-derived exosomes in ascites compared to serum.

The observation that the nuclear envelope of MN is unstable and prone to collapse, exposing its contents to the cell cytoplasm, prompted the idea that this event allows nuclear content to be loaded into exosomes (Hatch et al., 2013). As described in other sections, tetraspanins play a role in shuffling content into MVBs, which eventually gives rise to the secreted exosomes (Pols and Klumperman, 2009). In this study, CD63 was found to surround the MN envelope and load nuclear contents into exosomes. The role of CD63 in exosome cargo loading is further supported by a recent finding that CD63 interacts with the RNA binding protein, Y-box protein I, to load miR-223 into exosomes (Shurtleff et al., 2016). CD63 does not contain a canonical DNA binding domain. For this reason, it was hypothesize that CD63 may interact with DNA binding proteins, such as histones, to indirectly load gDNA into exosomes. The presence of a complex containing CD63—Histone H2B-gDNA in the experiments suggests that this could play an important role in the loading of DNA into exosomes.

Despite the growing interest in exosome biology, little is known about the biological function of nExos. Recently, Takahashi et al. (Takahashi et al., 2017) reported that gDNA is present in exosomes and that inhibiting total exosome secretion promotes cell senescence by stimulating the cGAS/STING inflammatory pathway. In recipient cells, it was demonstrated that T cell-derived extracellular vesicles containing genomic and mitochondrial DNA induced antiviral responses via the cGAS/STING cytosolic DNA-sensing pathway in dendritic cells (Torralba et al., 2018). In addition, it is known that MN can promote the activation of the cGAS/STING pathway, resulting in an inflammatory response in both senescent and cancer cells (Harding et al., 2017; Bakhoum et al., 2018).

Chromosomal instability has been observed to correlate with tumor metastasis, and a recent report suggested that this may be mediated by a cytosolic DNA response (Bakhoum et al., 2018). Inflammation is a key inducer of cancer progression by creating a nourishing tumor microenvironment (Lorusso and Riiegg, 2008). In this context, it is not surprising that the secretion of nuclear content by cancer cells via exosomes may promote metastasis by promoting a proinflammatory environment. It has been reported that plasma-derived exosomal DNA does not fully represent tumor CNV status due to the low concentration of tumor-derived exosomes (Kahlert et al., 2014). Consistent with this observation, the present data show that plasma-derived exosomes did not carry enough gDNA to obtain WGS libraries of sufficient complexity for accurate detection of CNV or single-nucleotide variation (SNV) (FIGS. 61 and 12). However, the data demonstrate that ascites-derived exosomes represent tumor CNV status. It is possible that the plasma exosome libraries show lower depth of coverage than the ascites exosomes or tumor. Several mutations related to DNA repair were found in matched tumor and ascites exosome DNA. These findings suggest that ascites nExos rather than serum nExo can be a reliable biomarker source to probe the tumor genome. This is particularly important in HGSC where distinct CNV signatures can serve as predictors of treatment response and ultimately clinical outcome (Macintyre et al., 2018). The ability to quickly isolate and probe ascites nExo CNV may allow clinicians to optimize therapy regimens without the direct biopsy of tumors.

### II. EXOSOMES

The terms “microvesicle” and “exosomes,” as used herein, refer to a membranous particle having a diameter (or largest dimension where the particles is not spheroid) of between about 10 nm to about 5000 nm, more typically between 30 nm and 1000 nm, and most typically between about 50 nm and 750 nm, wherein at least part of the membrane of the exosomes is directly obtained from a cell. Most commonly, exosomes will have a size (average diameter) that is up to 5% of the size of the donor cell. Therefore, especially contemplated exosomes include those that are shed from a cell.

Exosomes may be detected in or isolated from any suitable sample type, such as, for example, body fluids. As used herein, the term “isolated” refers to separation out of its natural environment and is meant to include at least partial purification and may include substantial purification. As used herein, the term “sample” refers to any sample suitable for the methods provided by the present invention. The sample may be any sample that includes exosomes suitable for detection or isolation. Sources of samples include blood, bone marrow, pleural fluid, peritoneal fluid, cerebrospinal fluid, urine, saliva, amniotic fluid, malignant ascites, broncho-alveolar lavage fluid, synovial fluid, breast milk, sweat, tears, joint fluid, and bronchial washes. In one aspect, the sample is a blood sample, including, for example, whole blood or any fraction or component thereof. A blood sample suitable for use with the present invention may be extracted from any source known that includes blood cells or components thereof, such as venous, arterial, peripheral, tissue, cord, and the like. For example, a sample may be obtained and processed using well-known and routine clinical methods (e.g., procedures for drawing and processing whole blood). In one aspect, an exemplary sample may be peripheral blood drawn from a subject with cancer.

Exosomes may also be isolated from tissue samples, such as surgical samples, biopsy samples, tissues, feces, and cultured cells. When isolating exosomes from tissue sources it may be necessary to homogenize the tissue in order to obtain a single cell suspension followed by lysis of the cells to release the exosomes. When isolating exosomes from tissue samples it is important to select homogenization and lysis procedures that do not result in disruption of the exosomes. Exosomes contemplated herein are preferably isolated from body fluid in a physiologically acceptable solution, for example, buffered saline, growth medium, various aqueous medium, etc.

Exosomes may be isolated from freshly collected samples or from samples that have been stored frozen or refrigerated. In some embodiments, exosomes may be isolated from cell culture medium. Although not necessary, higher purity exosomes may be obtained if fluid samples are clarified before precipitation with a volume-excluding polymer, to remove any debris from the sample. Methods of clarification include centrifugation, ultracentrifugation, filtration, or ultrafiltration. Most typically, exosomes can be isolated by numerous methods well-known in the art. One preferred method is differential centrifugation from body fluids or cell culture supernatants. Exemplary methods for isolation of exosomes are described in (Losche et al., 2004; Mesri and Altieri, 1998; Morel et al., 2004). Alternatively, exosomes may also be isolated via flow cytometry as described in (Combes et al., 1997).

One accepted protocol for isolation of exosomes includes ultracentrifugation, often in combination with sucrose density gradients or sucrose cushions to float the relatively low-density exosomes. Isolation of exosomes by sequential differential centrifugations is complicated by the possibility of overlapping size distributions with other microvesicles or macromolecular complexes. Furthermore, centrifugation may provide insufficient means to separate vesicles based on their sizes. However, sequential centrifugations, when combined with sucrose gradient ultracentrifugation, can provide high enrichment of exosomes.

Isolation of exosomes based on size, using alternatives to the ultracentrifugation routes, is another option. Successful purification of exosomes using ultrafiltration procedures that are less time consuming than ultracentrifugation, and do not require use of special equipment have been reported. Similarly, a commercial kit is available (EXOMIR™, Bioo Scientific) which allows removal of cells, platelets, and cellular debris on one microfilter and capturing of vesicles bigger than 30 nm on a second microfilter using positive pressure to drive the fluid. However, for this process, the exosomes are not recovered, their RNA content is directly extracted from the material caught on the second microfilter, which can then be used for PCR analysis. HPLC-based protocols could potentially allow one to obtain highly pure exosomes, though these processes require dedicated equipment and are difficult to scale up. A significant problem is that both blood and cell culture media contain large numbers of nanoparticles (some non-vesicular) in the same size range as exosomes. For example, some miRNAs may be contained within extracellular protein complexes rather than exosomes; however, treatment with protease (e.g., proteinase K) can be performed to eliminate any possible contamination with “extraexosomal” protein.

In another embodiment, cancer cell-derived exosomes may be captured by techniques commonly used to enrich a sample for exosomes, such as those involving immunospecific interactions (e.g., immunomagnetic capture). Immunomagnetic capture, also known as immunomagnetic cell separation, typically involves attaching antibodies directed to proteins found on a particular cell type to small paramagnetic beads. When the antibody-coated beads are mixed with a sample, such as blood, they attach to and surround the particular cell. The sample is then placed in a strong magnetic field, causing the beads to pellet to one side. After removing the blood, captured cells are retained with the beads. Many variations of this general method are well-known in the art and suitable for use to isolate exosomes. In one example, the exosomes may be attached to magnetic beads (e.g., aldehyde/sulphate beads) and then an antibody is added to the mixture to recognize an epitope on the surface of the exosomes that are attached to the beads.

As used herein, analysis includes any method that allows direct or indirect visualization of exosomes and may be in vivo or ex vivo. For example, analysis may include, but not limited to, ex vivo microscopic or cytometric detection and visualization of exosomes bound to a solid substrate, flow cytometry, fluorescent imaging, and the like.

It should be noted that not all proteins expressed in a cell are found in exosomes secreted by that cell. For example, calnexin, GM130, and LAMP-2 are all proteins expressed in MCF-7 cells but not found in exosomes secreted by MCF-7 cells (Baietti et al., 2012). As another example, one study found that 190/190 pancreatic ductal adenocarcinoma patients had higher levels of GPC1+ exosomes than healthy controls (Melo et al., 2015).

A. Exemplary Protocol for Collecting Exosomes from Cell Culture

On Day 1, seed enough cells (e.g., about five million cells) in T225 flasks in media containing 10% FBS so that the next day the cells will be about 70% confluent. On Day 2, aspirate the media on the cells, wash the cells twice with PBS, and then add 25-30 mL base media (i.e., no PenStrep or FBS) to the cells. Incubate the cells for 24-48 hours. A 48 hour incubation is preferred, but some cells lines are more sensitive to serum-free media and so the incubation time should be reduced to 24 hours. Note that FBS contains exosomes that will heavily skew NanoSight results.

On Day 3/4, collect the media and centrifuge at room temperature for five minutes at 800 ×g to pellet dead cells and large debris. Transfer the supernatant to new conical tubes and centrifuge the media again for 10 minutes at 2000 ×g to remove other large debris and large vesicles. Pass the media through a 0.2 μm filter and then aliquot into ultracentrifuge tubes (e.g., 25×89 mm Beckman Ultra-Clear) using 35 mL per tube. If the volume of media per tube is less than 35 mL, fill the remainder of the tube with PBS to reach 35 mL. Ultracentrifuge the media for 2-4 hours at 28,000 rpm at 4° C. using a SW 32 Ti rotor (k-factor 266.7, RCF max 133,907). Carefully aspirate the supernatant until there is roughly 1-inch of liquid remaining. Tilt the tube and allow remaining media to slowly enter aspirator pipette. If desired, the exosomes pellet can be resuspended in PBS and the ultracentrifugation at 28,000 rpm repeated for 1-2 hours to further purify the population of exosomes.

Finally, resuspend the exosomes pellet in 210 μL PBS. If there are multiple ultracentrifuge tubes for each sample, use the same 210 μL PBS to serially resuspend each exosomes pellet. For each sample, take 10 μL and add to 990 μL H₂O to use for nanoparticle tracking analysis. Use the remaining 200 μL exosomes-containing suspension for downstream processes or immediately store at −80° C.

B. Exemplary Protocol for Extracting Exosomes from Serum Samples

First, allow serum samples to thaw on ice. Then, dilute 250 μL of cell-free serum samples in 11 mL PBS; filter through a 0.2 μm pore filter. Ultracentrifuge the diluted sample at 150,000 ×g overnight at 4° C. The following day, carefully discard the supernatant and wash the exosomes pellet in 11 mL PBS. Perform a second round of ultracentrifugation at 150,000 ×g at 4° C. for 2 hours. Finally, carefully discard the supernatant and resuspend the exosomes pellet in 100 μL PBS for analysis.

### III. DIAGNOSIS, PROGNOSIS, AND TREATMENT OF DISEASES

Detection, isolation, and characterization of cancer cell-derived exosomes, using the methods of the invention, is useful in assessing cancer prognosis and in monitoring therapeutic efficacy for early detection of treatment failure that may lead to disease relapse. In addition, cancer cell-derived exosomes analysis according to the invention enables the detection of early relapse in presymptomatic patients who have completed a course of therapy. This is possible because the presence of cancer cell-derived may be associated and/or correlated with tumor progression and spread, poor response to therapy, relapse of disease, and/or decreased survival over a period of time. Thus, enumeration and characterization of cancer cell-derived exosomes provides methods to stratify patients for baseline characteristics that predict initial risk and subsequent risk based upon response to therapy.

Accordingly, in another embodiment, the invention provides a method for diagnosing or prognosing cancer in a subject. Cancer cell-derived exosomes isolated according to the methods disclosed herein may be analyzed to diagnose or prognose cancer in the subject. As such, the methods of the present invention may be used, for example, to evaluate cancer patients and those at risk for cancer. In any of the methods of diagnosis or prognosis described herein, either the presence or the absence of one or more indicators of cancer, such as a genomic mutation or cancer-specific exosomes surface marker, or of any other disorder, may be used to generate a diagnosis or prognosis.

In one aspect, a blood sample is drawn from the patient and cancer cell-derived exosomes are detected and/or isolated as described herein. Analysis may then be performed to determine the number and characterization of cancer cell-derived exosomes in the sample, and from this measurement, the number of cancer cell-derived exosomes present in the initial blood sample may be determined. The number of cancer cell-derived exosomes may be determined by cytometric or microscopic techniques to visually quantify and characterize the exosomes. Cancer cell-derived exosomes may be detected and quantifies by other methods known in the art (e.g., ELISA).

In various aspects, analysis of a subject's cancer cell-derived exosomes number and characterization may be made over a particular time course in various intervals to assess a subject's progression and pathology. For example, analysis may be performed at regular intervals such as one day, two days, three days, one week, two weeks, one month, two months, three months, six months, or one year, in order to track the level and characterization of cancer cell-derived exosomes as a function of time. In the case of existing cancer patients, this provides a useful indication of the progression of the disease and assists medical practitioners in making appropriate therapeutic choices based on the increase, decrease, or lack of change in cancer cell-derived exosomes. Any increase, be it 2-fold, 5-fold, 10-fold or higher, in cancer cell-derived exosomes over time decreases the patient's prognosis and is an early indicator that the patient should change therapy. Similarly, any increase, be it 2-fold, 5-fold, 10-fold or higher, indicates that a patient should undergo further testing such as imaging to further assess prognosis and response to therapy. Any decrease, be it 2-fold, 5-fold, 10-fold or higher, in cancer cell-derived exosomes over time shows disease stabilization and a patient's response to therapy, and is an indicator to not change therapy. For those at risk of cancer, a sudden increase in the number of cancer cell-derived exosomes detected may provide an early warning that the patient has developed a tumor thus providing an early diagnosis. In one embodiment, the detection of cancer cell-derived exosomes increases with the staging of the cancer.

In any of the methods provided herein, additional analysis may also be performed to characterize cancer cell-derived exosomes to provide additional clinical assessment. For example, in addition to image analysis and bulk number measurements, PCR techniques may be employed, such as multiplexing with primers specific for particular cancer markers to obtain information such as the type of tumor from which the cancer cell-derived exosomes originated, metastatic state, and degree of malignancy. Additionally, DNA or RNA analysis, proteome analysis, or metabolome analysis may be performed as a means of assessing additional information regarding characterization of the patient's cancer.

For example, the additional analysis may provide data sufficient to make determinations of responsiveness of a subject to a particular therapeutic regime, or for determining the effectiveness of a candidate agent in the treatment of cancer. Accordingly, the present invention provides a method of determining responsiveness of a subject to a particular therapeutic regime or determining the effectiveness of a candidate agent in the treatment of cancer by detecting/isolating cancer cell-derived exosomes of the subject as described herein and analyzing said cancer cell-derived exosomes. For example, once a drug treatment is administered to a patient, it is possible to determine the efficacy of the drug treatment using the methods of the invention. For example, a sample taken from the patient before the drug treatment, as well as one or more samples taken from the patient concurrently with or subsequent to the drug treatment, may be processed using the methods of the invention. By comparing the results of the analysis of each processed sample, one may determine the efficacy of the drug treatment or the responsiveness of the patient to the agent. In this manner, early identification may be made of failed compounds or early validation may be made of promising compounds.

Certain aspects of the present invention can be used to prevent or treat a disease or disorder based on the presence of genetic mutations found in genomic DNA isolated from exosomes. Other aspects of the present invention provide for treating a patient with exosomes that express a recombinant protein or with a recombinant protein isolated from exosomes. Other aspects of the present invention provide for diagnosing a disease based on the presence of cancer cell-derived exosomes in a patient sample.

The term “subject” as used herein refers to any individual or patient to which the subject methods are performed. Generally the subject is human, although as will be appreciated by those in the art, the subject may be an animal. Thus other animals, including mammals, such as rodents (including mice, rats, hamsters, and guinea pigs), cats, dogs, rabbits, farm animals (including cows, horses, goats, sheep, pigs, etc.), and primates (including monkeys, chimpanzees, orangutans, and gorillas) are included within the definition of subject.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of chemotherapy, immunotherapy, or radiotherapy, performance of surgery, or any combination thereof.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the invasiveness of a tumor, reduction in the growth rate of the cancer, or prevention of metastasis. Treatment of cancer may also refer to prolonging survival of a subject with cancer.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

The terms “contacted” and “exposed,” when applied to a cell, are used herein to describe the process by which a therapeutic agent are delivered to a target cell or are placed in direct juxtaposition with the target cell. To achieve cell killing, for example, one or more agents are delivered to a cell in an amount effective to kill the cell or prevent it from dividing.

An effective response of a patient or a patient's “responsiveness” to treatment refers to the clinical or therapeutic benefit imparted to a patient at risk for, or suffering from, a disease or disorder. Such benefit may include cellular or biological responses, a complete response, a partial response, a stable disease (without progression or relapse), or a response with a later relapse. For example, an effective response can be reduced tumor size or progression-free survival in a patient diagnosed with cancer.

Treatment outcomes can be predicted and monitored and/or patients benefiting from such treatments can be identified or selected via the methods described herein.

Regarding neoplastic condition treatment, depending on the stage of the neoplastic condition, neoplastic condition treatment involves one or a combination of the following therapies: surgery to remove the neoplastic tissue, radiation therapy, and chemotherapy. Other therapeutic regimens may be combined with the administration of the anticancer agents, e.g., therapeutic compositions and chemotherapeutic agents. For example, the patient to be treated with such anti-cancer agents may also receive radiation therapy and/or may undergo surgery.

For the treatment of disease, the appropriate dosage of a therapeutic composition will depend on the type of disease to be treated, as defined above, the severity and course of the disease, the patient's clinical history and response to the agent, and the discretion of the attending physician. The agent is suitably administered to the patient at one time or over a series of treatments.

Therapeutic and prophylactic methods and compositions can be provided in a combined amount effective to achieve the desired effect. A tissue, tumor, or cell can be contacted with one or more compositions or pharmacological formulation(s) comprising one or more of the agents, or by contacting the tissue, tumor, and/or cell with two or more distinct compositions or formulations. Also, it is contemplated that such a combination therapy can be used in conjunction with chemotherapy, radiotherapy, surgical therapy, or immunotherapy.

Administration in combination can include simultaneous administration of two or more agents in the same dosage form, simultaneous administration in separate dosage forms, and separate administration. That is, the subject therapeutic composition and another therapeutic agent can be formulated together in the same dosage form and administered simultaneously. Alternatively, subject therapeutic composition and another therapeutic agent can be simultaneously administered, wherein both the agents are present in separate formulations. In another alternative, the therapeutic agent can be administered just followed by the other therapeutic agent or vice versa. In the separate administration protocol, the subject therapeutic composition and another therapeutic agent may be administered a few minutes apart, or a few hours apart, or a few days apart.

A first anti-cancer treatment (e.g., exosomes that express a recombinant protein or with a recombinant protein isolated from exosomes) may be administered before, during, after, or in various combinations relative to a second anti-cancer treatment. The administrations may be in intervals ranging from concurrently to minutes to days to weeks. In embodiments where the first treatment is provided to a patient separately from the second treatment, one would generally ensure that a significant period of time did not expire between the time of each delivery, such that the two compounds would still be able to exert an advantageously combined effect on the patient. In such instances, it is contemplated that one may provide a patient with the first therapy and the second therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below a first anti-cancer therapy is “A” and a second anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present invention to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine,plicomycin, gemcitabien, navelbine, farnesyl-protein tansferase inhibitors, transplatinum, and pharmaceutically acceptable salts, acids, or derivatives of any of the above.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that additional immunotherapies may be used in combination or in conjunction with methods of the invention. In the context of cancer treatment, immunotherapeutics, generally, rely on the use of immune effector cells and molecules to target and destroy cancer cells. Rituximab (Rituxan®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include CD20, carcinoembryonic antigen, tyrosinase (p9′7), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDLL binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Publication Nos. 20140294898, 2014022021, and 20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUIDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Patent No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5844905, 5885796 and International Patent Application Nos. WO1995001994 and WO1998042752; all incorporated herein by reference, and immunoadhesins such as described in U.S. Pat. No. 8329867, incorporated herein by reference.

In some embodiment, the immune therapy could be adoptive immunotherapy, which involves the transfer of autologous antigen-specific T cells generated ex vivo. The T cells used for adoptive immunotherapy can be generated either by expansion of antigen-specific T cells or redirection of T cells through genetic engineering (Park, Rosenberg et al. 2011). Isolation and transfer of tumor specific T cells has been shown to be successful in treating melanoma. Novel specificities in T cells have been successfully generated through the genetic transfer of transgenic T cell receptors or chimeric antigen receptors (CARs) (Jena, Dotti et al. 2010). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. CARs have successfully allowed T cells to be redirected against antigens expressed at the surface of tumor cells from various malignancies including lymphomas and solid tumors (Jena, Dotti et al. 2010).

In one embodiment, the present application provides for a combination therapy for the treatment of cancer wherein the combination therapy comprises adoptive T-cell therapy and a checkpoint inhibitor. In one aspect, the adoptive T-cell therapy comprises autologous and/or allogenic T cells. In another aspect, the autologous and/or allogenic T cells are targeted against tumor antigens.

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present invention, chemotherapy, radiotherapy, hormonal therapy, gene therapy, immunotherapy, and/or alternative therapies.Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present invention to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present invention to improve the treatment efficacy.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Materials and Methods

Pan-cancer TCGA ploidy analysis. Ploidy data from TCGA samples were downloaded from the Pan-Cancer TCGA project (available on the world wide web at cell.com/pb-assets/consortium/pancanceratlas/pancani3/index.html; accessed 8 Sep. 2016).

Cell lines and tissue culture. The human ovarian cancer cell lines were obtained from the American Type Culture Collection (ATCC) and the University of Texas MD Anderson Cancer Center Characterized Cell Line Core Facility. Cell lines were routinely identified via short tandem repeat DNA profiling carried out by the Characterized Cell Line Core Facility at MD Anderson. Primary FTE cells were a gift from J. Liu from the Department of Pathology at MD Anderson. For all cell lines, mycoplasma testing was done using the ATCC PCR Universal Mycoplasma Detection Kit (30-1012K). OVCAR-8 cells were cultured in HyClone RPMI 1640 medium (SH30027.01, GE Healthcare Life Sciences) supplemented with 15% fetal bovine serum (FBS) (Sigma-Aldrich) and 0.2% gentamicin (50146970, Thermo Fisher Scientific). OVCAR-5 cells were cultured in Dulbecco's modified Eagle's medium (DMEM; 10-013-CV; Corning) supplemented with 10% FBS and 0.2% gentamicin. FTE cells were cultured in medium 199 with MCDB 105 (1:1) with 10% FBS and 0.2% gentamicin. All cells were grown in humidified incubators kept at 37° C. with 5% CO₂.

Isolation of exosomes. The isolation method for exosomes that was used in this study followed the standard methods of the International Society of Extracellular Vesicles (Witwer et al., 2013). Cell lines described above were grown in the corresponding medium described above until 60 to 70% confluence was achieved. Cells were then washed twice with phosphate-buffered saline (PBS) and grown in corresponding medium containing 1% exosome-free FBS (EXO-FBS-250A-1, System Biosciences) plus 0.1% gentamicin for 48 hours. For exosome isolation, the harvested medium underwent serial centrifugation. First, the medium was spun down at 500g for 10 min to pellet any floating cells. Next, the supernatant was collected and spun down at 2000g for 30 min to pellet any residual cell debris. The resulting supernatant was then filtered through a 0.22-μm filter (SCGPUO5RE and SLGP033RS, Millipore-Sigma) to remove any remaining large vesicles. After filtration, the medium was spun down at 40,000 rpm for 2 hours at 4° C. in an ultracentrifuge (Optima XE, Beckman Coulter) with a Ti 45 fixed-angle rotor (339160, Beckman Coulter). The resulting exosome pellet was resuspended in 2 ml of PBS and then spun again at 40,000 rpm for 2 hours at 4° C. The final pellet was resuspended in PBS and stored at 4° C. Exosome concentration and quantity were determined using NTA with a NanoSight NS300 instrument (Malvern Panalytical).

Isolation and purification of exosomes from patient serum and plasma. To isolate exosomes from serum and ascites obtained from patients with ovarian cancer, 200 μl of each patient's serum or ascites sample was collected and stored at 4° C. The serum was diluted with an equal volume of PBS and then centrifuged at 2000g for 30 min at 4° C. The supernatant was transferred to clean tubes without disturbing the pellet and centrifuged at 12,000g for 45 min at 4° C. The supernatant was then transferred to fresh tubes and diluted with a large volume of PBS (about 3 ml). After filtration with a 0.22-μm filter, the supernatant was transferred to ultracentrifuge tubes and ultracentrifuged at 40,000 rpm for 2 hours at 4° C. as described above. The supernatant was then discarded, and the exosome pellet was resuspended in 3 ml of PBS. This was followed by another 40,000 rpm ultracentrifugation spin for 70 min at 4° C. The final pellet was resuspended in PBS. To further purify patient-derived exosomes from abundant protein aggregates present in both serum and ascites, size exclusion chromatography was used as described previously (Boing et al., 2014). Exosome-enriched fractions were identified by both NTA and SDS-polyacrylamide gel electrophoresis (SDS-PAGE) methods (FIGS. 11A-11D).

Exosome staining for imaging flow cytometry and analysis. For quantitative analysis and imaging flow cytometry of exosomes, the method described by Erdbrugger et al. (2014) using an Amnis ImageStreamX MarkII analyzer was adapted. Briefly, 3×10⁹ to 5×10⁹ particles were isolated from either tissue culture cells, mouse serum and ascites, or serum obtained from patients with ovarian cancer and resuspended in PBS. Exosomes were placed in a sterile 1.5-ml Eppendorf tube and stained with CellMask Green Plasma Membrane (CMG) stain (1:250 dilution; C37608, Thermo Fisher Scientific) for 30 min at 37° C., protected from light. Exosomes were then washed to remove excess CMG by adding 2 ml of PBS and pelleted by ultracentrifugation at 40,000 rpm for 2 hours at 4° C. The supernatant was discarded, and the exosome pellet was gently resuspended in 100 to 150 μl of PBS. For antibody staining of exosomes, CMG-stained exosomes were incubated with fluorophore-conjugated primary antibodies described below for 1 hour with gentle agitation at room temperature (RT), protected from light. After 1 hour, the exosomes were washed by placing them in 2 ml of PBS and pelleting by ultracentrifugation at 40,000 rpm for 2 hours at 4° C. The resulting pellet was then resuspended in 100 to 150 μl of PBS.

To stain for dsDNA, CMG-stained exosomes were co-stained with DRAQ5 (62254, Thermo Fisher Scientific) at either 1:50 dilution for biofluid exosome samples or 1:250 dilution for cell culture samples and were lightly agitated at RT for 1 hour, protected from light. Samples were then placed on ice, and images of single exosomes were detected using the 60× objective of an Amnis ImageStreamX MarkII analyzer. The ImageStream was equipped with five lasers [200 mW 405 nm, 100 mW 488 nm, 200 mW 561 nm, 150 mW 642 nm, 70 mW 785 nm (SSC)], and all lasers were used at maximum power with the instrument set for 7 μm core diameter (low speed). Particle size was estimated using Invitrogen Flow Cytometry Submicron Particle Size Reference Beads (F13839, Thermo Fisher Scientific), allowing exclusion of particles <150 nm based on the signal detected from side scatter. Controls for all exosome analyses included detergent lysis controls, buffer controls without exosomes, reagents alone in buffer, and antibody-unstained samples. For detergent lysis controls, samples were incubated for 30 min at RT after adding the nonionic detergent NP-40 to a final concentration of 0.5%. All analyses, including gate placement and batch processing, were performed using the Amnis proprietary IDEAS and INSPIRE software packages at the South Campus Flow Cytometry and Cell Sorting Core, the University of Texas MD Anderson Cancer Center Flow Cytometry and Cellular Imaging Core Facility. Antibodies included the CD9 V450 mouse anti-human (1:100; 561326, BD Biosciences), Nesprin-2 (1:100; MA5-18075, Thermo Fisher Scientific) conjugated to Alexa 594, Lamin A/C 636 (SC-7292, Santa Cruz Biotechnology) conjugated to Alexa 594, Lamin B1 (1:100; 8982, Abcam), and Importin (1:100; ab2811, Abcam) conjugated to Alexa 594. Conjugation of antibodies for Nesprin-2, LaminBl, and importin to Alexa fluorophores was performed by using the Molecular Probes Antibody Labeling Kit (A20185 and A20181, Thermo Fisher Scientific).

IF experiments and MN quantification. Cells were plated at a density of 5×10⁴ cells on sterile, 0.17-mm-thick coverslips (12-548-A, Thermo Fisher Scientific) placed at the bottom of six-well plates. The next day, cells were treated for 24 hours with either vehicle control dimethyl sulfoxide (DMSO; 1:1000 dilution; D2650, Sigma-Aldrich), 20 μM olaparib (O-9201, LC Laboratories), or 10 nM topotecan (T2705-50MG, Sigma-Aldrich). After 24 hours, the cells were washed with PBS and refed with the appropriate cell culture medium to grow for another 48 hours, for a total time of 72 hours after initial plating on the coverslips.

The drug-treated cells were first washed three times with PBS, fixed with 4% paraformaldehyde (PFA; 50-980-487, Thermo Fisher Scientific) diluted in PBS, and incubated at RT for 15 min with light rocking. The PFA was discarded, cells were washed three times with PBS, and then incubated for 10 min at RT with 30 mM glycine (BP381-5, Thermo Fisher Scientific) diluted in PBS to quench any residual PFA. Next, the cells were washed three times with PBS and incubated with 0.2% Triton X-100 (BP151-500, Thermo Fisher Scientific) diluted in PBS for 10 min at RT with light shaking. Triton was discarded, and cells were placed in blocking buffer (10% goat serum and 1% bovine serum albumin in PBS, syringe-filtered with 0.22-μm filter) for 1 hour at 4° C. Cells were next incubated overnight at 4° C. with Lamin A/C (1:1000; ab26300, Abcam and 1:200; sc-5275, Santa Cruz Biotechnology), CD63 (1:1000; ab1318, Abcam), EEA1 (1:500; ab109110, Abcam), LAMP1 (1:100; ab108597, Abcam), and LC3B (1:200; ab64781, Abcam) antibodies diluted in blocking buffer. After primary antibody incubation, cells were washed three times with PBS and incubated with secondary antibodies Alexa Fluor 488 goat anti-mouse immunoglobulin G (IgG) (1:200; 115-545-003, Jackson ImmunoResearch) and Alexa Fluor 596 goat anti-rabbit (1:200; 111-586-047, Jackson ImmunoResearch) as well as DAPI (1:1000; D9542-1MG, Sigma-Aldrich) diluted in blocking buffer and incubated for 30 min at RT while protected from light. Last, cells on coverslips were then washed four times with PBS and mounted on slides using ProLong Diamond Antifade Mountant (P36961, Thermo Fisher Scientific) and imaged with a Leica fluorescence microscope (Leica DM4000 M LED; Leica Microsystems) or an Andor Revolution XDi WD Spinning Disk Confocal microscope. For MN quantification, the number of MN in each 40× field was counted using 10 fields per sample.

Confocal imaging. Cells were plated at a density of 2×10⁴ cells onto glass coverslips in six-well plates, placed back into the incubator, and allowed to grow for 48 to 72 hours. The growth medium was removed, and the cells were washed once with PBS and subsequently fixed with 3.7% formaldehyde for 15 min at RT. Cells were then permeabilized with 0.1% Triton X-100 for 5 min, washed two times, and blocked with 10% normal goat serum for 1 hour at 37° C. Primary antibodies CD63 (ab1318, Abcam), CD9 (ab97999, Abcam), CD81 (ab35026, Abcam), and Lamin A (ab26300 and ab8980, Abcam) were diluted in 1% normal goat serum and applied for 2 hours at 37° C. Primary antibodies were removed, cells were washed three times for 5 min with PBS, and incubated with Alexa-conjugated secondary antibodies diluted in Dulbecco's PBS for 1 hour at 37° C. Secondary antibodies were removed, coverslips were washed three times for 5 min, and mounted onto glass slides using Vectashield Hardset with DAPI (H-1500, Vector Labs). Images were acquired using a Zeiss LSM 710 confocal microscope with a 63x objective.

Western blotting. For Western blotting, harvested cells and exosomes were lysed with radioimmunoprecipitation assay buffer (RIPA) [25 mM tris (pH 7.5), 150 mM NaCl, 0.1% SDS, 0.5% sodium deoxycholate, 1% Triton X-100] supplemented with single-use phosphatase and protease inhibitors (78442, Thermo Fisher Scientific). Protein concentration was determined using the Micro BCA Protein Assay Kit (23235, Thermo Fisher Scientific) according to the manufacturer's protocol. For all Western blots, we used 5 to 10 μg of total cell or exosome lysate diluted with 2× Laemmli sample buffer (1610737, Bio-Rad), which was loaded onto 10% SDS denaturing polyacrylamide gels. Protein was transferred to a nitrocellulose membrane, blocked with 5% nonfat dry milk (AB10109-01000, AmericanBio) in tris-buffered saline with 0.1% Tween 20 (TBS-T) for 1 hour at RT, and incubated with the indicated antibodies diluted in 5% milk in TBS-T overnight at 4° C. with light agitation. The next day, the membrane was washed three times with TBS-T for 10 min with light agitation. After the third wash, the membrane was incubated with species-specific secondary antibodies conjugated to horseradish peroxidase (NA931V and NA934V; GE Healthcare) and diluted 1:2500 in 5% milk in TBS-T for 1 hour at RT with light agitation. The membrane was then washed three times in TBS-T and finally developed using Western Lightning Plus ECL (NEL105001EA; PerkinElmer) on x-ray film (F-BX57, Phenix). For reprobing of Western blots, membranes were stripped with Restore PLU.S. Western Blot Stripping Buffer (46430, Thermo Fisher Scientific), reblocked in 5% milk TBS-T, and incubated with primary antibody. In this study, the following primary antibodies and dilutions were used: CD63 (1:3000; EXOAB-CD63A-1, System Biosciences), TSG101 (1:500; ab30871, Abcam), Alix (1:500; SC-53538, Santa Cruz Biotechnology), GRP94 (1:500; SC-393402, Santa Cruz Biotechnology), Lamin A (1:1000; ab26300, Abcam), and Histone H2B (1:500; ab1790, Abcam).

Plasmid transfections. For transient transfection of cells for live cell imaging, a method similar to the one described above was used with some modifications. Briefly, cells were plated at a density of 3×10⁵ to 4×10⁵ cells per six-well plate so that cells reached 90% confluence the next day. For transfections, Lipofectamine 2000 (11668500, Thermo Fisher Scientific) was complexed with 1 μg of the plasmid mEmerald-CD9-10 (54029, Addgene), as described above. At 48 hours after transfection, mEmerald-CD9 expression was verified by using fluorescent microscopy, and cells were used for subsequent live cell imaging experiments.

Cloning of mCherry-LaminA/C into a lentiviral vector. The mCherry-LaminA/C fusion protein was cloned from the parental plasmid mCherry-LaminA-C-18 (55068, Addgene) using restriction enzyme digestion with Nhe I (FD0973, Thermo Fisher Scientific) and Bam HI (FD0054, Thermo Fisher Scientific). The parental lentiviral vector pCDH-CMV-MCS-EFla-Puro (CD510B-1, System Biosciences) was also digested with Nhe I and Bam HI. One microgram of each parental plasmid was used for the restriction enzyme digestions. Digestion products were run on a 1% agarose gel made with 1× TBE buffer (1.0 M tris, 0.9 M boric acid, and 0.01 M EDTA; 15581-028, Invitrogen UltraPure). The desired digestion products were cut and gel-purified using the GeneJET Gel Extraction Kit (K0692, Thermo Fisher Scientific) according to the manufacturer's protocol. mCherry-LaminA/C was ligated into pCDH-CMV-MCS-EF1α-Puro using T4 ligase (M0202S, NEB) for 12 hours at 16° C. according to the manufacturer's protocol. The ligation product was transformed into NEB Stable Competent Escherichia coli (C30401, NEB) according to the NEB protocol, plated on ampicillin plates, and grown at 37° C. for 12 hours. Colonies were picked and grown in LB medium at 37° C. (12795027, Thermo Fisher Scientific) supplemented with ampicillin (BP1760-5, Fisher BioReagents). Plasmid DNA was purified using the Qiagen Plasmid Plus Midi Kit (12945) according to the manufacturer's protocol.

Lentiviral production and cell transduction. For lentiviral production, human embryonic kidney (HEK) 293T cells (grown in DMEM, 10% FBS, and 0.2% gentamicin) were transfected using Lipofectamine 2000 (11668500, Thermo Fisher Scientific) with 10 μg of lentiviral plasmid (see below for plasmids used), along with 5 μg of psPAX2 (12260, Addgene) and 2.5 μg of pMD2.G (12259, Addgene) lentiviral helper plasmids. Cell medium containing newly generated virus was collected 48 and 72 hours after transfection, pooled, centrifuged to clear any cell debris, and syringe-filtered using a 0.45-μm filter (190-2545, Thermo Fisher Scientific). For infection, cells were plated at 50% confluence in six-well plates and incubated with 2 ml of newly produced virus along with polybrene at a 1:1000 dilution (sc-134220, Santa Cruz Biotechnology) for 24 hours. Cells were then refed with regular medium and allowed to grow. The cells with vectors containing drug selection markers such as puromycin (A11138-03, Gibco) were exposed to puromycin for 48 hours after initial lentiviral infection. Surviving cells were expanded and used for subsequent in vitro and in vivo experiments. Lentiviral vectors used in this study included pCDH-CMV-MCS-EF1α-Puro-mCherry-LaminA/C (see previous section), pCT-CD63-GFP (CYTO120-VA-1, System Biosciences), pLenti6-H2B-mCherry (89766, Addgene), and pLKO-CD63shRNA (SHCLNV-NM_001780, Sigma-Aldrich). For shRNA sequences, two different target sequences for human CD63 were used: (i) 5′-CCGGGCCTCGTGAAGAGTATCAGAACTCGAGTTCTGATACTCTTCACGAGGCTTT TT-3′ (SEQ ID NO: 1) and (ii) 5′-CCGGGCAAGGAGAACTATTGTCTTACTCGA GTAAGACAATAGTTCTCCTTGCTTTTT-3′ (SEQ ID NO: 2). The nontargeting shControl 5′-C CGGCCTAAGGTTAAGTCGCCCTCGCTCGAGCGAGGGCGACTTAACCTTAGGT TTTTG-3′ (SEQ ID NO: 3) was used.

Luciferase labeling of OVCAR-5 cells. To label cells with luciferase for in vivo imaging system (IVIS) imaging, cells were infected with the lentiviral vector pGreenFirel-CMV (TR011PA-1, System Biosciences), which coexpresses green fluorescent protein (GFP). Cells were sorted using FACSAria IIIu at the South Campus Flow Cytometry and Cell Sorting Core, MD Anderson Flow Cytometry and Cellular Imaging Core Facility for GFP positivity.

In vivo experiments. For the in vivo mouse experiments, female athymic (NCr-nude) mice were purchased from Taconic Biosciences. Mice were cared for in accordance with the American Association for Assessment and Accreditation of Laboratory Animal Care and the U.S. Public Health Service policy on Humane Care and Use of Laboratory Animals. All studies and experiments that were performed were supervised and approved by the MD Anderson Institutional Animal Care and Use Committee. Mice were 10 to 15 weeks old at the time of tumor intraperitoneal cell injections. OVCAR-5 luciferase—labeled cells (1×10⁶) were injected intraperitoneally into each mouse.

Injected cells were first grown in the indicated medium until they reached 70% confluence. The cells were then trypsinized (SH30042.01, GE Healthcare), washed twice with PBS, and resuspended in ice-cold Hanks' balanced salt solution (21-021-CV; Cellgro). To determine tumor cell uptake, mice were injected with 200 μl of luciferin (14.7 mg/ml) (LUCK-1G; GoldBio) and imaged using IVIS. Non-tumor-bearing mice were removed from the experiment. Mice were randomly assigned to the treatment groups. The primary investigator was not blinded to the allocation of each treatment group. At the end of the experiment, blood and ascites were collected from each tumor-bearing mouse, and exosomes were isolated.

Enrichment of MN fractions. Methods for isolation and purification of cultured cell MN were adapted from Damaraju et al. (Damaraju et al., 2010) with the following modifications: Harvested OVCAR-5 cells were scraped from 15-cm tissue cultures using ice-cold PBS supplemented with protease and phosphatase inhibitors and pelleted by centrifugation at 500g for 10 min at 4° C. The supernatant was carefully discarded, and the cell pellet was gently resuspended in 1 ml of ice-cold cell lysis buffer obtained from the Active Motif ChIP-IT Express Chromatin Immunoprecipitation Kit supplemented with protease and phosphatase inhibitors provided by the kit (53008, Active Motif). Cells were incubated in lysis buffer on ice for 30 min and then carefully pipetted into a 1-ml Dounce homogenizer. 20 to 25 strokes in the Dounce homogenizer were used to release intact cell nuclei from the rest of the cell components. To ensure successful cell lysis, a small aliquot of the cell suspension was stained with DAPI (1:1000) and imaged with our fluorescence microscope (FIG. 4A). Lysed cells were then spun at 2400g at 4° C. to pellet cell nuclei. Supernatant from this step, which contained MN, was kept, and pelleted cell nuclei were discarded. Cell supernatant was loaded to a sucrose gradient (84097-1KG, Sigma-Aldrich) prepared in PBS by using fractions indicated by Damaraju et al. (2010). The fraction corresponding to 25% sucrose was collected and mixed with an equal volume of ice-cold PBS. The fraction was centrifuged at 13,500g at 4° C. to pellet the MN, the supernatant was discarded, and MN were resuspended in ice-cold PBS with protease and phosphatase inhibitors. To confirm the presence of MN, an aliquot of the suspension was stained with DAPI and analyzed with a fluorescence microscope (FIG. 3A). The purified fraction was either sent for MS analysis or stored at −80° C.

Immunoprecipitation experiments. For IP analysis, whole-cell lysates were extracted following the manufacturer's protocol provided by the Universal Magnetic Co-IP Kit (54002, Active Motif), and 1000 μg of protein was immunoprecipitated using 2.5 of antibody. The prepared samples were used for Western blot and DNA staining by agarose gel electrophoresis.

DNase I treatment. Samples were treated with DNase I (1 unit/μl; E1011-A, Zymo Research) with DNase digestion buffer (E1011-1-4, Zymo Research) and incubated at 37° C. for 45 min. Subsequently, 5 μl of EDTA solution (1861274, Thermo Fisher Scientific) was added, and the samples were heated at 65° C. for 5 min.

Human tumor samples and MN quantification. High-grade ovarian tumor samples were obtained from MD Anderson Tissue Bank under an approved institutional review board protocol, and written consent was obtained for the use of patient samples for research. The details regarding the quality control for the samples obtained from CHTN can be found on the world wide web at chtn.org/quality.html. For MN identification and quantification, the Vectra Polaris platform (PerkinElmer) was used, and inForm software (PerkinElmer) was used to systematically count MN. After tissue segmentation, a number of nuclei in views were calculated by the cell segmentation process, and MN was defined as over 0.85 roundness and less than approximately 3 μm size (95 pixels in 96 dpi images), as shown in FIG. 3D.

Time-lapse cell imaging. Fluorescently labeled cells were plated onto sterilized four-chamber 0.170-mm glass-bottom slides (80427, Ibidi) at a density of 1×10⁴ cells per well. The following day, cells were imaged with an Andor Revolution XDi WD Spinning Disk Confocal microscope on a humidified stage, which was kept at 37° C. with 5% CO₂ to simulate optimal growing conditions. Hepes (10 mM) (25-060-Cl; Corning) buffer was added to the culture medium before imaging to maintain a stable pH throughout imaging. Cells were imaged every 5 min for movie S1 and every 10 min for movie S2 using a 63× silicon immersion objective lens for 12 hours. Images and movies were analyzed with Imaris Image Analysis Software (Bitplane, Oxford Instruments). For labeling of cell membranes, CellMask Deep Red (C10046, Thermo Fisher Scientific) was used according to the manufacturer's protocol.

Electron microscopy. Cells were prepared in the following manner: 2.5×10⁴ OVCAR-5 cells were plated on plastic 24-well tissue culture plates. Forty-eight hours later, cells were washed three times with PBS and fixed with 4% PFA with 0.1% of glutaraldehyde (G7651-10ML, Sigma-Aldrich) diluted in PBS, which was 0.22 μm syringe-filtered for 15 min at RT. Cells were then washed three times with PBS, and 2% glutaraldehyde was added to each well of cells. Twenty-four-well plate was then kept at 4° C. until ready for TEM processing at the MD Anderson Cancer Center High Resolution Electron Microscopy Facility. Samples were fixed with a solution containing 3% glutaraldehyde plus 2% PFA in 0.1 M cacodylate buffer (pH 7.3), then washed in 0.1 M sodium cacodylate buffer and treated with 0.1% Millipore-filtered cacodylate-buffered tannic acid, postfixed with 1% buffered osmium, and stained en bloc with 1% Millipore-filtered uranyl acetate. The samples were dehydrated in increasing concentrations of ethanol and then infiltrated with and embedded in LX-112 resin. The samples were polymerized in a 60° C. oven for approximately 72 hours. Ultrathin sections were cut in a Leica Ultracut microtome (Leica, Deerfield, Ill.), stained with uranyl acetate and lead citrate in a Leica EM stainer, and examined in a JEM 1010 transmission electron microscope (JEOL, USA Inc., Peabody, Mass.) at an accelerating voltage of 80 kV. Digital images were obtained using the AMT Imaging System (Advanced Microscopy Techniques Corp., Danvers, Mass.).

For cryo-EM, 1×10¹⁰ to 5×10¹⁰ exosomes were isolated from either ovarian cancer cells or patient plasma and ascites and resuspended in 50 μl of PBS. The samples were prepared for cryo-EM studies at the Baylor College of Medicine Cryo-Electron Microscopy Core Facility (BCM, Houston, Tex.). The grids (Quantifoil R2/1, Cu 200 mesh) were pretreated with a 45-s air-glow discharge immediately before vitrification. During vitrification, 3 μl of exosome sample was applied to a grid, blotted for 4 s, and subsequently plunged into liquid ethane using Vitrobot Mark IV (FEI Company, Hillsboro, Oreg.) set at RT and 100% humidity. The frozen grids were then imaged using a JEOL 2200FS microscope (JEOL) fitted with a post-column energy filter with a width of 30 eV. Before imaging, the microscope was carefully aligned to prevent any column-based distortion or astigmatism that can occur. Images were collected at magnifications of 25,000× and 40,000× with respective pixel sizes of 2.51 and 1.64 Å using a DE-20 camera (Direct Electron, San Diego, Calif.). Imaging was done with a dose rate of ˜30e−/Å 2/s using a 1-s exposure time with a capture rate of 24 frames/s. Gain and dark corrections were applied automatically to produce the final images used.

MS analysis. A total of about 20 to 40 μg of intact isolated exosomes from either OVCAR-5 cells or patient samples in addition to 40 μg of OVCAR-5 were quantified using the Micro BCA Protein Assay Kit (23235, Thermo Fisher Scientific) and sent to the MD Anderson Mass Spectrometry core. Proteins were acetone-precipitated (5:1) overnight at −20° C. and digested with 200 to 500 ng of modified trypsin (sequencing grade; Promega, Madison, Wis.) in the presence of RapiGest (Waters, Milford, Mass.) for 18 hours at 37° C. The resulting peptides were analyzed by high-sensitivity liquid chromatography—MS/MS on an Orbitrap-Fusion mass spectrometer (Thermo Fisher Scientific, Waltham, Mass.). Proteins were identified by searching the fragment spectra in the SwissProt (EBI) protein database using Mascot (version 2.6.2; Matrix Science, London, UK). Typical search settings were mass tolerances: 10 ppm (parts per million) precursor, 0.8d fragments; variable modifications, methionine sulfoxide, pyro-glutamate formation; up to two missed cleavages. False discovery rate estimates were from Proteome Discoverer (version 2.2, Thermo Fisher Scientific).

Library preparation and sequencing. Exosome DNA was isolated from 1×10¹⁰ to 5×10¹⁰ exosomes from either tissue culture cells or bodily fluids with the System Biosciences XCF Exosomal DNA Isolation Kit (XCF200A-1, Systems Biosciences) following the manufacturer's protocol. gDNA from either cultured cells or tumor tissue was isolated using the Qiagen DNeasy Blood and Tissue Kit (69506, Qiagen) according to the manufacturer's protocol. DNA concentration was determined using both the NanoDrop 2000 Spectrophotometer (Thermo Fisher Scientific) and the Agilent 2100 Bioanalyzer Kit (5067-150, Agilent Technologies) following the manufacturer's protocol.

WGS next-generation sequencing (NGS) libraries were constructed either using the KAPA HyperPlus Kit (Roche Holding AG) or the Zymo Pico Methyl-Seq Kit (Zymo Research), depending on the source of the DNA sample. The KAPA kit provides a relatively unbiased enzymatic fragmentation method, which allows balanced coverage and low-input polymerase chain reaction (PCR)-free DNA NGS library construction (Ring et al., 2017). dsDNA (Onco-NoPCR) (300 ng) in 0.5 mM EDTA was added with the appropriate amount of conditioning solution to the enzymatic fragmentation step. The dsDNA was then incubated with the fragmentation enzyme for 6 min at 37° C. The fragmented samples were then end-repaired and A-tailed, and Illumina-indexed adapters were ligated onto the ends of the dsDNA. A two-sided AmpureXP bead selection protocol (0.55×/0.8×/2.5×) was used to select for an average NGS library size of approximately 481 base pairs (bp).

The Zymo kit protocol is a post-bisulfite adapter tagging NGS library protocol and is relatively unbiased as a hybridization-based NGS library generation method. This protocol was modified to exclude the bisulfite DNA fragmentation step, extend the initial adapter tagging extension step from 8 to 30 min, and extend the two PCR extension steps from 1 to 6 min. ExDNA (extracellular DNA) (100 ng) was used to generate a NGS library with this Zymo kit. Six cycles of PCR were used during the final index adapter addition step. A two-sided AmpureXP bead selection protocol (0.55×/0.8×/1.2×) was used to select for an average NGS library size of approximately 473 bp.

The NGS libraries were quantified using a PicoGreen-based assay (Qubit, Thermo Fisher Scientific), and for quality control purposes, their size distribution profiles were determined using a Bioanalyzer instrument (Agilent Technologies Inc.) The NGS libraries were then sequenced on a NovaSeq 6000 instrument (Illumina, San Diego, Calif.) using paired-end 2×150-bp reads (UCSF Functional Genomics Core, University of California, San Francisco, San Francisco, Calif.) or a HiSeq 4000 instrument (Illumina, San Diego, Calif.) using paired-end 2×100-bp reads (IGM Genomics Center, University of California, San Diego, La Jolla, Calif.).

Bioinformatics analysis. Regarding cell line samples, the fastq files were first quality-controlled using FastQC (version 0.11.4) and adapters were trimmed using Trim Galore (version 0.4.1). The trimmed fastq files were then mapped to GRCh38 (GCA_000001405.15) using Bowtie2 (version 2.2.7) (Langmead and Salzberg, 2012) and duplicated, sorted, and indexed using Samtools (version 1.3). The OncoNoPCR sample was sequenced to an average depth of 23×, and the ExDNA was sequenced to an average depth of 20×. Copy number alterations were called using cnvkit-0.9.6a0-py27_2. The batch WGS method was used after calculating the sequence-accessible coordinates from the reference genome. CNVs were then called using the following command: cnvkit.py batch—method wgs *sort.bam -n -f . . . /GCA_000001405.15_GRCh38_no_alt_analysis_set.fna—access access-excludes.GRCh38.bed—output-reference my_flat_reference.cnn -p 12 —scatter—diagram -d cnv_data/. Adjacent regions with the same copy number calls were merged using the cnvkit.py call—filter option. Copy number alterations were considered to be overlapping if they contained at least a 30% reciprocal overlap using Bedtools (version 2.25.0). Figures were made using cnvkit's scatter function and circos-0.69-3. Regarding clinical samples, fastq files are aligned to the reference genome (human Hg19) using Burrows-Wheeler Alignment tool (BWA) with three mismatches, with two in the first 40 seed regions for sequences less than 100 bp. The aligned BAM files are subjected to mark duplication, realignment, and recalibration using Picard and GATK before any downstream analyses. Somatic mutations were called using MuTect, and indels were called using Pindel. DNA copy number analysis is conducted using HMMcopy following circular binary segmentation (CBS). DNA from blood cells was used as the reference in all of the above analyses. Mutation and indel calls were filtered using thresholds of ≥20 reads covering the called event for plasma, ascites, or tumor tissues and ≥20 reads for blood cells. An allele frequency cutoff of 0.2 was also applied to the mutation and indel data.

Statistical analysis. All statistical analyses of in vitro and in vivo experiments were done using GraphPad Prism 7 and the SPSS software program (version 24.0, IBM Corporation). To determine whether differences between the two groups were significant, a two-tailed Student's t test (equal variance) was used. For these analyses, a P value of <0.05 was considered statistically significant. Results were presented as the mean ±SD.

### Example 1

### Liquid Biopsy Using nExo

Exosomes were isolated from ovarian cancer cell lines, normal human fallopian epithelial tube cells and bio-fluids from mouse and human samples. Ovarian cancer is characterized for having high genomic instability (FIG. 1A). To determine the purity of exosomes, nanoparticle tracking system, immunoblotting assay and cryo-transmission electron microscopy were used. To analyze the content of exosomes, protein mass spectrometry and imaging flow cytometry analysis was performed. First, it was determined that mass spectrometry analyses for ovarian cancer cell exosomes revealed that 12.5% of proteins are derived from the cell nucleus sub-population (FIGS. 1B-1F and 6A-6D). Additionally, using a novel imaging flow cytometry technique which enables the identification and quantification of single exosomes, it was determined that 10% of exosomes carried gDNA (FIGS. 7D, 7E, 7G, and 7H). Conversely, normal cells secreted only 0.2% of nExo (FIGS. 11, 2B, and 2C). It was then hypothesized that the prevalence of gDNA is affected by genotoxic drugs. Treating cells with either the PARP inhibitor olaparib or topoisomerase inhibitor topotecan increased the proportion of nExo secreted from ovarian cancer cells (FIGS. 2G and 2H). In addition, PARP proteins exist in ovarian cancer cell exosomes and 80.4% of the exosomes co-localized with gDNA (FIG. 14). Using in vivo preclinical models, serum from ovarian cancer bearing mice contained a higher number of nExo than non-tumor controls, and this population was increased in response to treatment of genotoxic drugs (FIGS. 3A, 3B, 3G, 3J, and 3K). Furthermore, plasma and ascites from ovarian cancer patients contain around 0.3% of nExo, which include nuclear proteins and gDNA (FIGS. 3A, 3B, 3G, 3J, and 3K).

### Example 2

### Ovarian Cancer Exosomes Contain Nuclear Content

Despite the prevalence of gDNA in cancer-derived exosomes, the relationship between genomic instability and the abundance of nExo has not been elucidated. To address this problem, The Cancer Genome Atlas (TCGA) was screened to determine which tumor types have the highest number of chromosomal duplications and are thus the most genomically unstable (FIG. 1A). Of the 25 tumor types investigated, high-grade serous ovarian cancer (HGSC) ranked fourth highest in median ploidy and the genome was characterized as highly unstable, which has been associated with poor clinical outcomes (Matulonis et al., 2016).

Using HGSC preclinical models, the purity of the exosome isolation approach was tested with cryo-electron microscopy (cryo-EM), nanoparticle tracking analysis (NTA), and immunoblotting assays (FIGS. 1B-1D and 7A-7C). To determine whether the exosomes carried nuclear proteins, a mass spectrometry (MS) analysis was performed on the exosomal fractions. In the exosomes isolated from OVCAR-5 (OVCAR-5exo) cells, an HGSC cell line, 201 nuclei-associated proteins and 17 chromosome-associated proteins were detected, and 12.5% of the total number of detected proteins were nuclear-derived (FIGS. 1E and 1F).

On the basis of these findings, whole-genome sequencing (WGS) was used to compare CNV between the DNA from OVCAR-5 cells and exosomes (FIG. 1G). The CNVs were quite similar between the cell and exosome DNA (FIG. 1H). While some studies have shown that gDNA is present in exosomes (Balaj et al., 2011; Thakur et al., 2014), the subpopulation of exosomes that contain DNA and the amount of exosomes that contain DNA are unclear. To address this question, the Amnis Image Stream X, MkII flow cytometer, which can detect single exosomes as previously described, was used (Erdbrugger et al., 2014). This methodology was adapted to analyze nExo by first staining the exosomes with the CellMask plasma membrane stain and loading onto the flow analyzer. Beads of various sizes were used as reference to set the gates for the exosome population (FIG. 1I, left upper panel, and FIGS. 7D-7F). The particles with a side-scatter (SSC) aspect ratio close to 1 were specifically selected to exclude exosome aggregates from the analysis (FIG. 7G). To confirm that the particles detected were bound by lipid membranes, exosomes were treated with 0.5% NP-40 to disrupt the membrane. This almost completely abolished the presence of detected exosomes (FIG. 7H). Furthermore, exosome concentration positively correlated (R2=0.968) with the particles detected by the analyzer (FIG. 7I). These results demonstrate the successful detection of exosomes by the imaging flow cytometry method. In addition, this method can be used for exosome analysis without the procedure of exosome isolation because it detected exosomes by using cell culture medium (FIG. 7J).

To identify the presence of gDNA and to quantify the percentage of DNA-positive exosomes, the exosomes were stained with DRAQ5, a dye that preferentially binds double-stranded DNA (dsDNA) and is cell permeant to allow quantification of the percentage of DNA-positive exosomes (FIG. 1I). In addition, the single-particle imaging flow cytometry method allowed quantification of subpopulations of DNA-positive exosomes that were also positive for other exosome protein markers, such as tetraspanins (FIGS. 1I and 8A). CD9 and CD63, well-documented exosome markers, were abundantly detected in both DNA-positive and DNA-negative exosomes (FIG. 8A). To confirm the intra-exosomal localization of DNA, exosomes were treated with deoxyribonuclease (DNase) Ito degrade DNA attached to the outer membrane of exosomes. There was approximately a 50% reduction in DNA-positive exosomes after DNase I treatment (FIG. 8B). These results suggest that gDNA is present on both the surface and the inside of exosomes. In addition to gDNA, exosomes were also tested for the presence of Lamin A/C, a nuclear envelope protein that was present in the OVCARS-exo MS data (FIGS. 1E and 1F). Lamin A/C was also detectable by flow cytometry (FIG. 1J). This result was further validated by detection of Lamin A/C from isolated exosomes via Western blot (FIG. 8C). Contrary to gDNA, Lamin A/C was not as abundant in OVACRS-exo, but more than 50% of DNA-positive exosomes were also positive for Lamin A/C (FIGS. 1J and 8D). Other nuclear proteins, such as importin, Nesprin-2, and Lamin B1, were also detected in DNA-positive exosomes via flow cytometry in OVCARS-exo, but their abundance was more variable (FIG. 8E).

### Example 3

### Induction of MN Increases the Population of DNA-Carrying Exosomes

Similar to the presence of gDNA in cancer-derived exosomes, micronuclei (MN) are also more prevalent in cancer cells due to their inherent genomic instability (Fenech et al., 2011). On the basis of this idea, the inventors investigated whether any relationship existed between the presence of MN and nExo. First, the baseline number of cells containing MN was determined in primary healthy fallopian tube epithelial (FTE) cells compared to ovarian cancer cell lines. In primary FTE cells, considered to be the cell of origin for many HGSCs (Ng and Barker, 2015), the prevalence of MN-containing cells was 1% (FIG. 2A). In contrast, about 4% of ovarian cancer cells contained MN (FIG. 2A). Comparing the amount of nExo secreted by healthy and cancerous cells, cancer cells secreted a significantly higher population of nExo (FTEexo, 0.12±0.04%; OVCAR-5exo, 8.26±1.62%; and OVCAR-8exo, 8.25±2.61%) (FIGS. 2B and 2C). These data suggest that cells with high genomic instability and therefore increased MN, such as cancer cells, secrete a larger number of nExos.

To further test this hypothesis, ovarian cancer cells were treated with the genotoxic drugs topotecan (10 nM) or olaparib (20 μM) to induce the formation of MN. Upon treatment with either drug, the number of MN increased significantly (FIGS. 2D and 2E). Similarly, the population of exosomes containing gDNA (FIGS. 2F, 2G, 9A, and 9B) and nuclear proteins (FIG. 9C) was also increased after treatment with these drugs. These results indicate that inducing genomic instability increases MN production and subsequently increases nExo abundance.

To validate these findings in vivo, the OVCAR-5 ovarian cancer model, which represents disseminated peritoneal disease, was used. As shown in the schematic diagram (FIG. 3A), each tumor-bearing mouse was treated with the maximum tolerated dose of topotecan (7.5 mg/kg) and euthanized 48 hours later (Mitra et al., 2015). Tumor growth in each mouse was confirmed by in vivo imaging (FIG. 3B). To determine whether topotecan could induce MN formation, as observed in vitro, tumor nodules were harvested, sectioned, and stained with hematoxylin and eosin (H&E) and examined via bright light and by immunofluorescence (IF) microscopy (FIGS. 3C and 3D). H&E staining confirmed the presence of MN after topotecan treatment (FIG. 3C). To calculate the number of MN, multiple IF images of tumor slides were taken, and MN were systematically counted by the Vectra—inForm Image Analysis System (PerkinElmer) (FIG. 3D). There was a trend toward increased MN-positive cell percentages in the topotecan-treated group versus vehicle control, although this percentage was not statistically significant (FIG. 3E).

To measure differences in the amount of nExos produced after each treatment, exosomes were isolated from serum and ascites of tumor-bearing mice from each group, and their purity was confirmed by cryo-EM, NTA, and Western blotting for CD63 (FIGS. 3F-3H) and quantified by imaging flow cytometry. Tumor-bearing mice generally had significantly more nExos in their serum than did the non-tumor-bearing mice (P=0.008) (FIG. 3I). Comparing topotecan-treated mice with vehicle control, a significant increase of nExos was observed in ascites (P=0.028) but no difference in the serum nExos (FIGS. 3J and 3K). These results indicate that tumors produce more nExos and that genotoxic drugs promote MN formation in vivo, increasing the amount of nExo release.

### Example 4

### MN and nExo Share Content and Interact in Live Cells

Next, the relative nuclear protein abundance was compared between MN and nExos. MN from OVCAR-5 cells were isolated via sucrose gradient ultracentrifugation, and fractions enriched for MN were washed and submitted for MS (FIG. 4A) (Damaraju et al., 2010). MN enrichment was confirmed with 4′,6-diamidino-2-phenylindole (DAPI) staining and IF (FIG. 4A). MS analysis of the MN fraction revealed substantial similarity between OVCAR-5 MN and nExo protein content, with 127 proteins overlapping between the two cellular compartments (FIGS. 4B and 4C). Some of these shared nuclear proteins included Lamin A/C and Histone H2B as well as some exosome markers such as heat shock proteins (FIG. 4C).

Next, whether MN and exosome-associated proteins interact within cells was determined. Confocal imaging of exosome markers such as CD63, CD9, and CD81 revealed colocalization of these proteins either in the nuclear envelope or inside the MN of ovarian cancer cells. To further explore whether this interaction occurred in live cells, time-lapse imaging of cells overexpressing fluorescently tagged nuclear proteins and tetraspanins was carried out. Both CD9 and CD63 actively interacted with MN in OVCAR-5 cells. These findings suggest that both MN and nExos share a high degree of nuclear content and that these structures actively interact within living cells.

### Example 5

### Cargo of Disrupted MN is Loaded into Exosomes

The envelope of MN is known to be unstable, and upon its collapse, MN contents including gDNA are exposed to the cell cytoplasm (Hatch et al., 2013). Therefore, whether this collapse can induce the loading of MN contents into exosomes was explored. Consistent with previous findings, confocal imaging of ovarian cancer cells showed that some MN were not surrounded by their nuclear envelope, suggesting MN collapse. Of interest, tetraspanin proteins, which can serve as markers for multivesicular bodies (MVBs), often surrounded the MN with either partial or total nuclear envelope collapse. These results were verified by transmission electron microscopy (TEM) (FIGS. 5A-5C). This revealed that, similar to the confocal observations, ovarian cancer cells contained collapsed MN (FIG. 5A and 10).

To address the mechanism behind nuclear content loading into exosomes, it was hypothesized that collapsing MN directly interact with the molecular machinery for exosome biosynthesis. Exosomes are initially formed from the intraluminal vesicles of early endosomes and are then stored in MVBs (Raposo and Stoorvogel, 2013). These vesicles are then released as exosomes to the extracellular space by fusion of MVBs with the plasma membrane (Raposo and Stoorvogel, 2013). TEM revealed that MVBs were located near MN and early endosomes, and thus could directly interact with collapsing MN (FIGS. 5C and 5D). Further, confocal analysis showed that intact and collapsed MN were both positive for EEA1, the early endosome marker. However, a recent report indicates that extracellular DNA can be secreted outside of the exosomal pathway via amphisomes (Jeppesen et al., 2019). In accordance with this, MN also contained LC3B, an amphisome and autophagosome marker, revealed by immunostaining. In contrast, MN contained less LMAP-1, the late endosome/lysosome marker, compared to EEA1 and LC3B. These results suggest that gDNA can be secreted in an exosome-dependent or exosome-independent manner, in which collapsing MN can interact with either early endosomes or amphisomes.

To further explore the molecular mechanism driving MN content loading into exosomes, tetraspanins, such as CD63, were focused on, as they are involved in exosome/MVB cargo loading (Pols and Klumperman, 2009). To determine whether tetraspanins are necessary for loading MN content into the endosomes and ultimately into nExos, CD63 was knocked down in ovarian cancer cell lines with short hairpin RNA (shRNA). After confirmation of CD63 protein knockdown (FIG. 5D), changes in the number of nExos by flow cytometry was examined. Cells with CD63 knockdown secreted fewer nExos than control cells (FIG. 5E), indicating that CD63 plays an important role in loading nuclear content into nExos. To further explore the role of CD63 in nExo loading, immunoprecipitation (IP) experiments were performed. CD63 pulldown from OVCAR-5 cell lysates revealed an association with DNA and Histone H2B (FIGS. 5F and 5G). Thus, CD63 can create a complex of gDNA and nuclear proteins and may be important for DNA loading in exosomes.

### Example 6

### Detection of nExo in Clinical Samples

To explore the clinical relevance of nExo, exosomes were isolated from the plasma and ascites of patients with HGSC to characterize their exosomal contents (FIGS. 6A and 6B). Because of the abundant immunoglobulin contamination present in exosomes isolated by ultracentrifugation from human serum, these exosomes were purified using size exclusion chromatography and then MS proteomic analysis was performed (Boing et al., 2014). MS analysis demonstrated that, of the total proteins identified in patient-derived exosomes, 3.2 to 3.6% were nuclear proteins (FIGS. 6C, 6D, and 11A-11E). MN were also detected in human tumor tissues, with a prevalence of 1% micronucleated cells per tumor analyzed (FIGS. 6E, 6F, and 11F). Using flow cytometry, <1% of exosomes contained gDNA, which was consistent with the in vivo mouse model data (FIGS. 3I and 11G).

WGS analysis of advanced-stage HGSC patient samples revealed that 43 gene mutations were found in both tumor and nExos derived from ascites. Several of these mutated genes are involved in DNA repair, e.g., DROSHA, LIG4, MACROD2, SATB1, RASSF6, and BIRC2 (FIG. 6H) (Lu et al., 2018; Chapman et al., 2012; Sakthianandeswaren et al., 2018; Kaur et al., 2016; Iwasa et al., 2013; Adamson et al., 2012). In addition, based on the result of read counts, the vast majority of DNA in exosomes was genomic in origin rather than mitochondrial, and the mutation signature was similar (FIGS. 61 and 12A). Moreover, ascites exosomes have a CNV similar to the primary tumor, but plasma exosomes did not (FIGS. 6J and 12B-12D).

### * * *

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Adamson et al., A genome-wide homologous recombination screen identifies the RNA-binding protein RBMX as a component of the DNA-damage response. Nat. Cell Biol. 14, 318-328 (2012).
Baietti et al., Syndecan-syntenin-ALIX regulates the biogenesis of exosomes. Nat. Cell Biol. 14:677-685 (2012).
Bakhoum et al., Chromosomal instability drives metastasis through a cytosolic DNA response. Nature 553, 467-472 (2018).
Balaj et al., Tumour microvesicles contain retrotransposon elements and amplified oncogene sequences. Nat. Commun. 2, 180 (2011).
Bitler et al., PARP inhibitors: Clinical utility and possibilities of overcoming resistance. Gynecol. Oncol. 147, 695-704 (2017).
Böing et al., Single-step isolation of extracellular vesicles by size-exclusion chromatography. J. Extracell. Vesicles 3, 23430 (2014).
Chapman et al., Playing the end game: DNA double-strand break repair pathway choice. Mol. Cell 47, 497-510 (2012).
Combes et al., A new flow cytometry method of platelet-derived microvesicle quantitation in plasma, Thromb. Haemost., 77:220, 1997.
Crasta et al., DNA breaks and chromosome pulverization from errors in mitosis. Nature 482, 53-58 (2012).
Creemers et al., Topoisomerase I inhibitors: Topotecan and irenotecan. Cancer Treat. Rev. 20, 73-96 (1994).
Damaraju et al., Evidence for copurification of micronuclei in sucrose density gradient-enriched plasma membranes from cell lines. Anal. Biochem. 396, 69-75 (2010).
Erdbrugger et al., Imaging flow cytometry elucidates limitations of microparticle analysis by conventional flow cytometry. Cytometry A 85, 756-770 (2014).
Fenech, Cytokinesis-block micronucleus cytome assay. Nat. Protoc. 2, 1084-1104 (2007).
Fenech et al., Molecular mechanisms of micronucleus, nucleoplasmic bridge and nuclear bud formation in mammalian and human cells. Mutagenesis 26, 125-132 (2011).
Jagetia and Reddy, The grapefruit flavanone naringin protects against the radiation-induced genomic instability in the mice bone marrow: A micronucleus study. Mutat. Res. 519, 37-48 (2002).
Jeppesen et al., Reassessment of exosome composition. Cell 177, 428-445.e18 (2019).
Harding et al., Mitotic progression following DNA damage enables pattern recognition within micronuclei. Nature 548, 466-470 (2017).
Hatch et al., Catastrophic nuclear envelope collapse in cancer cell micronuclei. Cell 154, 47-60 (2013).
Iwasa et al., The RASSF6 tumor suppressor protein regulates apoptosis and the cell cycle via MDM2 protein and p53 protein. J. Biol. Chem. 288, 30320-30329 (2013).
Kahlert et al., Identification of double-stranded genomic DNA spanning all chromosomes with mutated KRAS and p53 DNA in the serum exosomes of patients with pancreatic cancer. J. Biol. Chem. 289, 3869-3875 (2014).
Kaur et al., Special AT-rich sequence-binding protein 1 (SATB1) functions as an accessory factor in base excision repair. J. Biol. Chem. 291, 22769-22780 (2016).
Kosaka et al., Versatile roles of extracellular vesicles in cancer. J. Clin. Invest. 126, 1163-1172 (2016).
Langmead and Salzberg, Fast gapped-read alignment with Bowtie 2. Nat. Methods 9, 357-359 (2012).
Lorusso and Rüegg, The tumor microenvironment and its contribution to tumor evolution toward metastasis. Histochem. Cell Biol. 130, 1091-1103 (2008).
Losche et al., Platelet-derived microvesicles transfer tissue factor to monocytes but not to neutrophils, Platelets, 15:109-115, 2004.
Lu et al., Drosha drives the formation of DNA:RNA hybrids around DNA break sites to facilitate DNA repair. Nat. Commun. 9, 532 (2018).
Macintyre et al., Copy number signatures and mutational processes in ovarian carcinoma. Nat. Genet. 50, 1262-1270 (2018).
Mateescu et al., Obstacles and opportunities in the functional analysis of extracellular vesicle RNA—An ISEV position paper. J. Extracell. Vesicles 6, 1286095 (2017).
Matulonis et al., Ovarian cancer. Nat. Rev. Dis. Primers. 2, 16061 (2016).
Melo et al., Glypican-1 identifies cancer exosomes and detects early pancreatic cancer. Nature 523:177-182, 2015.
Mesri and Altieri, Endothelial cell activation by leukocyte microparticles, J. Immunol., 161:4382-4387, 1998.
Mitra et al., In vivo tumor growth of high-grade serous ovarian cancer cell lines. Gynecol. Oncol. 138, 372-377 (2015).
Morel et al., Cellular microparticles: a disseminated storage pool of bioactive vascular effectors, Curr. Opin. Hematol., 11:156-164, 2004.
Ng and Barker, Ovary and fimbrial stem cells: Biology, niche and cancer origins. Nat. Rev. Mol. Cell Biol. 16, 625-638 (2015).
Pols and Klumperman, Trafficking and function of the tetraspanin CD63. Exp. Cell Res. 315, 1584-1592 (2009).
Raposo and Stoorvogel, Extracellular vesicles: Exosomes, microvesicles, and friends. J. Cell Biol. 200, 373-383 (2013).
Ring et al., A performance evaluation of Nextera XT and KAPA HyperPlus for rapid Illumina library preparation of long-range mitogenome amplicons. Forensic Sci. Int. Genet. 29, 174-180 (2017).
Sakthianandeswaren et al., MACROD2 deletions cause impaired PARP1 activity and chromosome instability in colorectal cancer. Oncotarget 9, 33056-33058 (2018).
Schwarzenbach et al., Cell-free nucleic acids as biomarkers in cancer patients. Nat. Rev. Cancer 11, 426-437 (2011).
Shurtleff et al., Y-box protein 1 is required to sort microRNAs into exosomes in cells and in a cell-free reaction. eLife 5, e19276 (2016).
Takahashi et al., Exosomes maintain cellular homeostasis by excreting harmful DNA from cells. Nat. Commun. 8, 15287 (2017).
Thakur et al., Double-stranded DNA in exosomes: A novel biomarker in cancer detection. Cell Res. 24, 766-769 (2014).
Théry et al., Exosomes: Composition, biogenesis and function. Nat. Rev. Immunol. 2, 569-579 (2002).
Torralba et al., Sánchez-Madrid, Priming of dendritic cells by DNA-containing extracellular vesicles from activated T cells through antigen-driven contacts. Nat. Commun. 9, 2658 (2018).
Valadi et al., Exosome-mediated transfer of mRNAs and microRNAs is a novel mechanism of genetic exchange between cells. Nat. Cell Biol. 9, 654-659 (2007).

Witwer et al., Standardization of sample collection, isolation and analysis methods in extracellular vesicle research. J. Extracell. Vesicles 2, 20360 (2013).

What is claimed is:

1. A method of diagnosing cancer in a patient, the method comprising:
(a) obtaining a body fluid sample from a patient;
(b) isolating an exosomes fraction of the body fluid sample; and
(c) assaying for the presence of nuclear proteins and/or genomic DNA in the exosomes fraction, wherein if nuclear proteins and/or genomic DNA is present, then the patient is diagnosed as having cancer.

2. The method of claim 1, further comprising quantifying the number of nuclear proteins and/or genomic DNA-containing exosomes in the patient.

3. The method of claim 2, wherein the number of nuclear proteins and/or genomic DNA-containing exosomes in the patient is higher than a reference number.

4. The method of claim 3, wherein the reference number is the number of nuclear proteins and/or genomic DNA-containing exosomes in a sample obtained from a healthy subject.

5. The method of any one of claims 2-4, further defined as a method of monitoring response to a therapy in a cancer patient, wherein if the number of nuclear proteins and/or genomic DNA-containing exosomes increases over time, then the patient is said to have had a positive response to the therapy.

6. The method of claim 5, wherein the therapy is a genotoxic therapy.

7. The method of claim 6, wherein the genotoxic therapy is a PARP inhibitor.

8. The method of claim 6, wherein the genotoxic therapy is a topoisomerase inhibitor.

9. The method of any one of claims 1-8, wherein the patient has not been previously diagnosed with cancer and the method is a method of early cancer detection.

10. The method of any one of claims 1-8, wherein the patient is in remission and the method is a method of detecting relapse.

11. The method of any one of claims 1-10, further comprising administering an anti-cancer therapy to the patient if the patient is diagnosed with cancer.

12. The method of any one of claims 1-11, wherein the body fluid sample is lymph, saliva, urine, cerebrospinal fluid, bone marrow aspirates, eye exudate/tears, or serum.

13. The method of any one of claims 1-12, wherein the cancer is a ovarian cancer, breast cancer, lung cancer, head & neck cancer, prostate cancer, esophageal cancer, tracheal cancer, brain cancer, liver cancer, bladder cancer, stomach cancer, pancreatic cancer, uterine cancer, cervical cancer, testicular cancer, colon cancer, rectal cancer or skin cancer.

14. The method of any one of claims 1-13, further comprising reporting the diagnosis of the patient.

15. The method of claim 14, wherein reporting comprises preparing a written or electronic report.

16. The method of claim 15, further comprising providing the report to the patient, a doctor, a hospital or an insurance company.

17. A method of identifying genetic alterations in a tumor, the method comprising:
(a) obtaining an ascites fluid sample from a patient having a tumor;
(b) isolating an exosomes fraction of the ascites fluid sample; and
(c) assaying for the presence of genetic alterations in a genomic DNA present in the exosomes fraction, wherein any genetic alternations present in the genomic DNA present in the exosomes fraction are identifies as genetic alternation in the tumor.

18. The method of claim 17, further comprising administering an anti-cancer therapy to the patient if the patient.

19. The method of claim 17 or 18, wherein the cancer is a ovarian cancer, breast cancer, lung cancer, head & neck cancer, prostate cancer, esophageal cancer, tracheal cancer, brain cancer, liver cancer, bladder cancer, stomach cancer, pancreatic cancer, uterine cancer, cervical cancer, testicular cancer, colon cancer, rectal cancer or skin cancer.

20. A method of treating a cancer in a subject comprising, identifying a subject as having a cancer in accordance with any one of claims 1-16 and administering an anti-cancer therapy to the subject.

21. The method of claim 20, wherein the anti-cancer therapy is a chemotherapy, a radiation therapy, a hormonal therapy, a targeted therapy, an immunotherapy or a surgical therapy.

22. The method of claim 20 or 21, wherein the subject is a human.

23. A method of selecting a cancer patient for treatment with a PARP inhibitor, the method comprising:
(a) obtaining a body fluid sample from a patient;
(b) isolating an exosomes fraction of the body fluid sample; and
(c) assaying for the presence of PARP protein in the exosomes fraction, wherein if PARP protein is present, then the patient is selected for treatment with the PARP inhibitor.

24. The method of claim 23, wherein the patient has ovarian cancer.

25. A method of monitoring response to a therapy in a cancer patient, the method comprising:
(a) obtaining a body fluid sample from a patient at two or more time points during the course of therapy;
(b) isolating an exosomes fraction of the body fluid samples; and
(c) assaying for the presence of nuclear proteins and/or genomic DNA in the exosomes fractions, wherein if the number of nuclear proteins and/or genomic DNA-containing exosomes increases over time, then the patient is said to have had a positive response to the therapy.

26. The method of claim 25, wherein the therapy is a genotoxic therapy.

27. The method of claim 26, wherein the genotoxic therapy is a PARP inhibitor.

28. The method of claim 26, wherein the genotoxic therapy is a topoisomerase inhibitor.

29. The method of any one of claims 25-28, further comprising administering a different anti-cancer therapy to the patient if the patient is not found to have had a positive response to the therapy.

30. The method of any one of claims 25-29, wherein the body fluid sample is lymph, saliva, urine, cerebrospinal fluid, bone marrow aspirates, eye exudate/tears, or serum.

31. The method of any one of claims 25-30, wherein the cancer is a ovarian cancer, breast cancer, lung cancer, head & neck cancer, prostate cancer, esophageal cancer, tracheal cancer, brain cancer, liver cancer, bladder cancer, stomach cancer, pancreatic cancer, uterine cancer, cervical cancer, testicular cancer, colon cancer, rectal cancer or skin cancer.

32. A method of analyzing exosomes, the method comprising:
(a) obtaining a fluid sample comprising exosomes;
(b) staining the exosomes with a plasma membrane stain; and
(c) analyzing the exosomes using imaging flow cytometry by selecting particles with a side-scatter aspect ratio of about one.

33. The method of claim 32, wherein the exosomes are isolated between steps (a) and (b).

34. The method of claim 32, wherein the exosomes are not isolated between steps (a) and (b).

35. The method of claim 32, wherein the method is a method of isolating exosomes.

36. The method of claim 32, wherein the plasma membrane stain is a CellMask plasma membrane stain.


SEQUENCE LISTING (3 sequences)

SEQ ID NO: 1 - DNA, 57 bp, Artificial Sequence
  Synthetic polynucleotide
ccgggcctcgtgaagagtatcagaactcgagttctgatactcttcacgaggcttttt

SEQ ID NO: 2 - DNA, 57 bp, Artificial Sequence
  Synthetic polynucleotide
ccgggcaaggagaactattgtcttactcgagtaagacaatagttctccttgcttttt

SEQ ID NO: 3 - DNA, 58 bp, Artificial Sequence
  Synthetic polynucleotide
ccggcctaaggttaagtcgccctcgctcgagcgagggcgacttaaccttaggtttttg